(19) United States
(12) Patent Application Publication
(10) Pub. No.: US 2004/0146870 A1
(43) Pub. Date: Jul. 29, 2004

(54) Systems and methods for predicting specific genetic loci that affect phenotypic traits

(76) Inventors: Guochun Liao, San Mateo, CA (US); Gary Allen Peltz, Redwood City, CA (US); Jonathan Andrew Usuka, Palo Alto, CA (US)

Correspondence Address: MORGAN, LEWIS & BOCKIUS, LLP., 3300 HILLVIEW AVENUE, PALO ALTO, CA, 94304, US

(21) Appl. No.: 10/352,846

(22) Filed: Jan. 27, 2003

(51) Int. Cl. C12Q 1/68; G06F 19/00; G01N 33/48; G01N 33/50

(52) U.S. Cl. 435/6; 702/20

(57) ABSTRACT

A database of genetic variations is analyzed to produce a haplotype map of the genome for strains of a single species. A computational method is used to rapidly map complex phenotypes onto the haplotype blocks within the haplotype map. The specific genetic locus regulating three different biologically important phenotypic traits in mice is identified using these systems and methods.

### 1. FIELD OF THE INVENTION

This invention pertains to systems and methods for predicting chromosomal regions that affect phenotypic traits.

### 2. BACKGROUND OF THE INVENTION

Identification of genetic loci that regulate susceptibility to disease has promised insight into pathophysiologic mechanisms and the development of novel therapies for common human diseases. Family studies clearly demonstrate a heritable predisposition to many common human diseases such as asthma, autism, schizophrenia, multiple sclerosis, systemic lupus erythematosus, and type I and type II diabetes mellitus. For a review, see Risch, Nature 405, 847-856, 2000. Over the last 20 years, causative genetic mutations for a number of highly penetrant, single gene (Mendelian) disorders such as cystic fibrosis, Huntington's disease and Duchene muscular dystrophy have been identified by linkage analysis and positional cloning in human populations. These successes have occurred in relatively rare disorders in which there is a strong association between the genetic composition of a genome of a species (genotype) and one or more physical characteristics exhibited by the species (phenotype).

It was hoped that the same methods could be used to identify genetic variants associated with susceptibility to common diseases in the general population. For a review, see Lander and Schork, Science 265, 2037-2048, 1994. Genetic variants associated with susceptibility to subsets of some common diseases such as breast cancer (BRCA-1 and -2), colon cancer (FAP and HNPCC), Alzheimer's disease (APP) and type II diabetes (MODY-1, -2, -3) have been identified by these methods, which has raised expectations. However, these genetic variants have a very strong effect in only a very limited subset of individuals suffering from these diseases (Risch, Nature, 405, 847-856, 2000).

Despite considerable effort, genetic variants accounting for susceptibility to common, non-Mendelian disorders in the general population have not been identified. Since multiple genetic loci are involved, and each individual locus makes a small contribution to overall disease susceptibility, it will be quite difficult to identify common disease susceptibility loci by applying conventional linkage and positional cloning methods to human populations. Mapping of disease susceptibility genes in human populations has also been hampered by variability in phenotype, genetic heterogeneity across populations, and uncontrolled environmental influences. The variable reports of linkage between the chromosome 1q42 region and systemic lupus erythematosus illustrate the difficulties encountered in human genetic studies. One group reported strong linkage between the 1q42 region (Tsao, J. Clin. Invest, 99, 725-731, 1997) and to microsatellite alleles of a gene (PARP) within that region (Tsao, J. Clin. Invest. 103, 1135-1140, 1999). In contrast, no evidence for association with the PARP microsatellite marker was noted (Criswell et al., J. Clin. Invest, June; 105, 1501-1502, 2000; Delrieu et al., Arthritis & Rheumatism 42, 2194-2197, 1999); and minimal (Mucenski, et al., Molecular & Cellular Biology 6, 4236-4243, 1986) or no linkage (Lindqvist, et al., Journal of Autoimmunity, March; 14, 169-178, 2000) to the 1q42 region was found in several other SLE populations analyzed. It is likely that additional tools and approaches will be needed to identify genetic factors underlying common human diseases.

Analysis of experimental murine genetic models of human disease biology should greatly facilitate identification of genetic susceptibility loci for common human diseases. Experimental murine models have the following advantages for genetic analysis: inbred (homozygous) parental strains are available, controlled breeding, common environment, controlled experimental intervention, and ready access to tissue. A large number of murine models of human disease biology have been described, and many have been available for a decade or more. Despite this, relatively limited progress has been made in identifying genetic susceptibility loci for complex disease using murine models. Genetic analysis of murine models requires generation, phenotypic screening and genotyping of a large number of intercross progeny. Using currently available tools, this is a laborious, expensive and time-consuming process that has greatly limited the rate at which genetic loci can be identified in mice, prior to confirmation in humans. For a review, see Nadeau and Frankel, Nature Genetics August; 25, 381-384, 2000.

The difficulties encountered in associating phenotypic variations, such as susceptibility to common diseases, with genetic variations gives rise to a need in the art for additional tools for identifying chromosomal regions that are most likely to contribute to quantitative traits or phenotypes. In view of this situation, it would be highly desirable to provide a technique for associating a phenotype with one or more specific genetic loci in the genome of an organism without reliance on time consuming techniques such as cross breeding experiments or laborious post-PCR manipulation.

### 3. SUMMARY OF THE INVENTION

The present invention provides computer systems and methods for associating a phenotype with one or more specific genetic loci in the genome of a single species. In the method, phenotypic differences between a plurality of organisms of the single species are correlated with variations and/or similarities in the respective genomes of the organisms. The invention first computes a haplotype map based on the polymorphisms in the plurality of organisms. The distribution of phenotypes associated with the species are then compared with the distribution of alleles in each haplotype block in the haplotype map in order to identify haplotype blocks within the haplotype map that potentially regulate or affect the phenotypes.

One aspect of the present invention provides a method of associating a phenotype exhibited by a plurality of different organisms of a single species with one or more specific loci in a genome of the single species. In the method, a haplotype block in a haplotype map is scored based on a correspondence between variations in a phenotypic data structure and variations in the haplotype block. In some embodiments, the phenotypic data structure represents a difference in the phenotype exhibited by the plurality of different organisms and the haplotype map comprises a plurality of haplotype blocks. Each haplotype block in the haplotype map represents a different portion of the genome. The scoring is performed for each haplotype block in the plurality of haplotype blocks in the haplotype map. This results in the identification of one or more haplotype blocks in the plurality of haplotype blocks having a better score than all other haplotype blocks in the plurality of haplotype blocks.

In some embodiments, a haplotype block in the plurality of haplotype blocks comprises a plurality of consecutive single nucleotide polymorphisms. In some embodiments, each single nucleotide polymorphism in the haplotype block is within a threshold distance of another single nucleotide polymorphism in the haplotype block. In some embodiments, this threshold distance is less than ten megabases or less than one megabase. In some embodiments, there is no limitation on the distance between SNPs in the haplotype block.

In some embodiments, a haplotype block in the plurality of haplotype blocks represents a plurality of haplotypes and less than a cutoff percentage of the haplotypes represented by the haplotype block appear only once in the haplotype block. In other words, no more than a cutoff percentage of the haplotypes in any given haplotype block are exhibited by only a single organism in the plurality of organisms. In some embodiments, the cutoff percentage is in a range between five percent and thirty percent.

Some embodiments of the invention further comprise the step of generating the haplotype map prior to the scoring. The haplotype map can be generated by a variety of different methods. In one such method, a candidate haplotype block is identified in a genotypic database. The candidate haplotype block has a plurality of consecutive single nucleotide polymorphisms. In some embodiments, each single nucleotide polymorphism in the candidate haplotype block is within a threshold distance of another single nucleotide polymorphism in the candidate haplotype block. In some embodiments, there is no limitation on the distance between the single nucleotide polymorphisms within a candidate haplotype block. A score is assigned to the candidate haplotype block. This identification and scoring is repeated until all possible candidate haplotype blocks in the genotype database have been identified, thereby creating a set of candidate haplotype blocks. Next, a candidate haplotype block having the highest score in the set of candidate haplotype blocks is selected for the haplotype maps. Then, the selected candidate haplotype block and each candidate haplotype block that overlays all or a portion of the selected candidate haplotype block is removed from the set of candidate blocks. The process of selecting a candidate haplotype block for the haplotype map and removing the selected block and all blocks that overlap the selected block from the set of undiscarded blocks is repeated until no candidate haplotype block remains in the set of candidate haplotype blocks. In this approach, the haplotype map comprises each candidate haplotype block that was selected from the set of candidate blocks. In some embodiments, the score is a number of single nucleotide polymorphisms in the candidate haplotype block divided by a square of the number of haplotypes represented by the block.

The present invention additionally provides methods for computing a score between variations in a haplotype block and variations in a phenotype exhibited by a plurality of different organisms of a single species. In some embodiments, such scoring comprises assigning a score S to the haplotype block wherein

$$S = - log ( \frac{\sum {D}_{intra}}{\sum \vert {D}_{inter} \vert} )$$

where

ΣDᵢₙₜᵣₐ is a summation of the differences in phenotypic values for organisms in the plurality of organism that share the same haplotype in the haplotype block, and

ΣDᵢₙₜₑᵣ is the summation of the differences in phenotypic values between organisms in the plurality of organisms that do not share the same haplotype in the haplotype block In some embodiments, such scoring comprises assigning a score S to the haplotype block wherein

$$S = ( \frac{\sum {D}_{intra}}{\sum \vert {D}_{inter} \vert} )$$

where ΣDᵢₙₜᵣₐ and ΣDᵢₙₜₑᵣ have the same meanings presented above. In some embodiments S is the negation, inverse, negated inverse, logarithm or negated logarithm of the ratio presented above. In some embodiments, ΣDᵢₙₜᵣₐ or ΣDᵢₙₜₑᵣ is raised to a power (e.g., ½, 2 or 10).

In some embodiments, the specific genetic locus in the one or more specific genetic loci identified by the systems and methods of the present invention has a length that is less than 0.5 of a megabase, between 0.5 of a megabase and 2.0 megabases, or less than 10 megabases. In some embodiments, the phenotype investigated by the systems and methods of the present invention is diabetes, cancer, asthma, schizopherenia, arthritis, multiple sclerosis, rheumatosis, an autoimmune disorder or a genetic disorder. In some embodiments, the phentotypic data structure is microarray expression data. In some embodiments, the single species studied using the methods of present invention is an animal (e.g., human or mouse), a plant, Drosophila, a yeast, a virus, or C. elegans. In some embodiments, the plurality of different organisms of the single species is between five and 1000 organisms.

In addition to providing methods for associating chromosomal regions in a single species with a phenotype exhibited by organisms of the single species, the systems and methods of the present invention provide ways to elucidate biological pathways in the single species. One such method for accomplishing this includes the step of (i) selecting a haplotype in the one or more haplotype blocks in the plurality of haplotype blocks obtained using the methods described above. The haplotype block from which the haplotype is selected has a better score than all or most other haplotype blocks in the plurality of haplotype blocks. A secondary haplotype map is generated for the single species using genotypic data for the organisms in the plurality of different organisms of the single species that are represented in the selected haplotype. Then, a haplotype block in the secondary haplotype map is scored. This score represents a correspondence between variations in the phenotypic data structure and variations in the selected haplotype block. The steps of selecting a haplotype block in the secondary haplotype map and scoring the selected haplotype block are repeated for each haplotype block in the secondary haplotype map, thereby identifying one or more secondary haplotype blocks having a better score than all other haplotype blocks in the secondary haplotype map. Then a biological pathway for the single species is constructed. This pathway includes (a) a locus in the haplotype block from the haplotype block from which the haplotype was selected and (b) a locus from the one or more secondary haplotype blocks that received a better score than other haplotype blocks.

In some embodiments, the phenotypic data structure represents measurements of a plurality of cellular constituents in the plurality of organisms. In some embodiments, the phenotype data structure comprises a phenotypic array for each organism in the plurality of organisms and each phenotypic array comprises a differential expression value for each cellular constituent in a plurality of cellular constituents in the organism represented by the phenotypic array. Each of the differential expression values, in turn represent a difference between (i) a native expression value of a cellular constituent in an organism in the plurality of organisms; and (ii) an expression value of the cellular constituent in the organism after the organism has been exposed to a perturbation. In some embodiments, the perturbation is a pharmacological agent. In some embodiments, the perturbation is a chemical compound having a molecular weight of less than 1000 Daltons.

In some embodiments of the present invention, an organism in the plurality of different organisms is a member of the single species, a cellular tissue derived from a member of the single species, or a cell culture derived from the member of the single species.

Another aspect of the present invention provides a computer program product for use in conjunction with a computer system. The computer program product comprises a computer readable storage medium and a computer program mechanism embedded therein. The computer program mechanism comprises a genotypic database, a phenotypic data structure, a haplotype map, and a phenotype/haplotype processing module. The genotypic database is for storing variations in genomic sequences of a plurality of different organisms of a single species. The phenotypic data structure represents a difference in a phenotype exhibited by the plurality of different organisms. The haplotype map comprises a plurality of haplotype blocks, each haplotype block in the haplotype map representing a different portion of the genome of the single species. The phenotype/haplotype processing module is for associating a phenotype exhibited by the plurality of different organisms with one or more specific genetic loci in the genome of the single species. The phenotype/haplotype processing module comprises a phenotype/haplotype comparison subroutine. The phenotype/haplotype comparison subroutine comprises

instructions for scoring a haplotype block in the haplotype map, this scoring representing a correspondence between variations in the phenotypic data structure and variations in the haplotype block; and

instruction is for re-executing the instructions for scoring for each haplotype block in the plurality of haplotype blocks in the haplotype map, thereby identifying one or more haplotype blocks in the plurality of haplotype blocks having a better score than all other haplotype blocks in the plurality of haplotype blocks.

Another aspect of the present invention provides a computer system for associating a phenotype exhibited by a plurality of different organisms with one or more specific genetic loci in the genome of a single species. The computer system comprises a central processing unit and a memory coupled to the central processing unit. The memory stores a genotypic database, a phenotypic data structure, a haplotype map, and a phenotype/haplotype processing module, each of which has the same functions as presented above.

### 4. BRIEF DESCRIPTION OF THE DRAWINGS

FIG. 1 illustrates a computer system for associating a phenotype with a haplotype block in a genome of an organism in accordance with one embodiment of the present invention.

FIG. 2 illustrates the processing steps for associating a phenotype with a haplotype block in a genome of an organism in accordance with one embodiment of the present invention.

FIGS. 3A, 3B and 3C illustrate select single nucleotide polymorphism (SNP) data and the haplotypes represented by the select SNP data.

FIGS. 4A and 4B illustrate select single nucleotide polymorphism (SNP) data and the haplotypes represented by the select SNP data.

FIG. 4C illustrates hypothetical quantitative phenotypic values for each of the strains represented in FIGS. 4A and 4B.

FIG. 5 illustrates the haplotype block structure on mouse chromosome 1 between 48 to 58 megabases where each column represents a different mouse strain (organism) and each row represents a SNP. The two possible SNP alleles are respectively represented by dark shading and light shading and ambiguous haplotypes (due to missing data) are not shaded.

FIG. 6A illustrates a representative haplotype block structure on chromosome 7 (22.7 Mb) constructed using A/J, 129, C57BL/6 and CAST/Ei strains in which each haplotype block is set off by horizontal lines.

FIG. 6B illustrates a comparison of haplotype blocks constructed respectively using three (A/J, 129 and C57BL/6) and thirteen Mus Musculus strains in which SNPs present at the bound of haplotype blocks are joined by lines.

FIG. 7A illustrates, using all SNPs on mouse chromosome 1, the percentage of the total number of SNPs included in haplotype blocks (squares) and the number of SNPs per block (diamonds) as a function of the number of mouse strains.

FIG. 7B illustrates, using all SNPs on mouse chromosome 1, the number of haplotypes per block as a function of the number of strains analyzed.

FIGS. 8A, 8B, and 8C illustrate computational mapping of phenotypic data onto haplotype blocks in accordance with one embodiment of the present invention.

FIG. 9 illustrates the correlation between MHC K haplotype and the structure of one predicted haplotype block on chromosome 17 where major alleles are indicated by dark shading, minor alleles are indicated by light shading, and the absence of shading indicates missing allelic data.

FIG. 10A illustrates the level of pulmonary Cyp1a1 gene expression for each inbred mouse strain.

FIG. 10B illustrates how the 79 SNPs in the haplotype block structure of the Ahr locus on chromosome 12 form three haplotype groups and how seven exonic SNPs (labeled a-g) result in an amino acid change in the protein.

FIG. 10C illustrate the amino acid changes in the Ahr protein for the three haplotype groups illustrated in FIG. 10B.

FIG. 11 illustrates the processing steps for reconstructing a biological pathway using the methods of the present invention.

Like reference numerals refer to corresponding parts throughout the several views of the drawings.

### 5. DETAILED DESCRIPTION OF THE INVENTION

The present invention is directed toward computer systems and methods for building a haplotype map based upon variations in the genomes of organisms of a single species. The present invention is further directed to computer systems and methods for identifying haplotype blocks within the haplotype map that potentially affect phenotypic traits associated with the species. This identification step is performed by evaluating how well a distribution of alleles within each haplotype block in the haplotype map match phenotypic data associated with the single species under study.

### 5.1 Overview of an Exemplary System

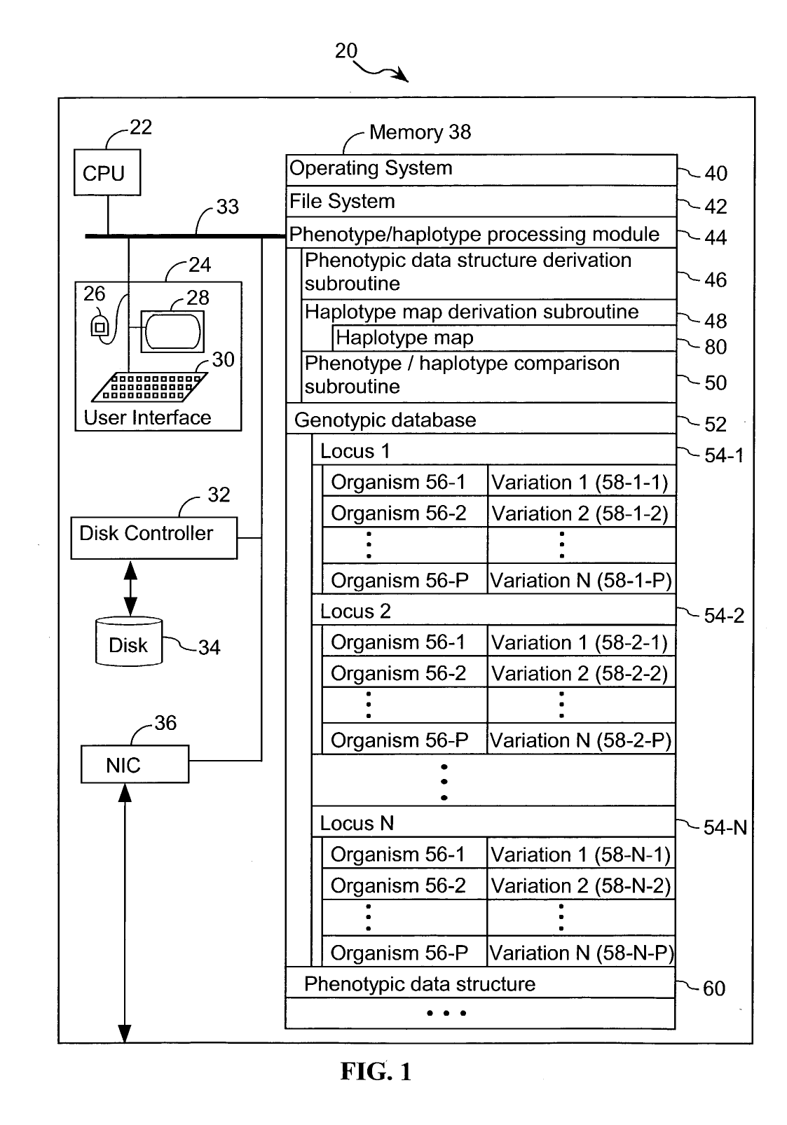

FIG. 1 shows a system 20 for associating a phenotype with one or more haplotype blocks in a genome of an organism.

System 20 preferably includes:

a central processing unit 22;

a main non-volatile storage unit 34, preferably including one or more hard disk drives, for storing software and data, the storage unit 34 typically controlled by disk controller 32;

a system memory 38, preferably high speed random-access memory (RAM), for storing system control programs, data, and application programs, including programs and data loaded from non-volatile storage unit 34; system memory 38 may also include read-only memory (ROM);

a user interface 24, including one or more input devices, such as a mouse 26 and a keypad 30, and a display 28;

an optional network interface card 36 for connecting to any wired or wireless communication network; and

an internal bus 33 for interconnecting the aforementioned elements of the system.

Operation of system 20 is controlled primarily by operating system 40, which is executed by central processing unit 22. Operating system 40 may be stored in system memory 38. In addition to operating system 40, a typical implementation of system memory 38 includes:

file system 42 for controlling access to the various files and data structures used by the present invention;

phenotype/haplotype processing module 44 for associating a phenotype with one or more haplotype blocks in a haplotype map;

genotypic database 52 for storing variations in genomic sequences of a plurality of organisms of a single species; and

phenotypic data structure 60 that includes measured differences in one or more phenotypic traits associated with the single species.

In a preferred embodiment, phenotype/haplotype processing module 44 includes:

a phenotypic data structure derivation subroutine 46 for deriving a phenotypic data structure that represents a variation in a phenotype between different organisms of a single species;

a haplotype map derivation subroutine 48 for generating a haplotype map 80 from variations in the genome of a plurality of organisms in a single species; and

a phenotype/haplotype comparison subroutine 50 for comparing the phenotypic array to the haplotype map 80 in order to identify haplotype blocks within the haplotype map 80 in which the distribution of alleles within the block matches the distribution of alleles exhibited by the species under study.

### 5.2 Exemplary Genotypic Databases

Information that is typically represented in genotypic database 52 is a collection of loci 54 within the genome of the single species. For each locus 54, organisms 56 for which genetic variation information is available are represented in database 52. For each represented organism 56, variation information 58 is provided. Variation information 58 is any form of genetic variation between organisms of a single species. Representative variation information 58 includes, but is not limited to, single nucleotide polymorphisms (SNPs), restriction fragment length polymorphisms (RFLPs), microsatellite markers, short tandem repeats, sequence length polymorphisms, and DNA methylation. Exemplary genotypic databases 52 are provided in Table 1.

TABLE 1
Exemplary Sources of Genotypic Databases

| Genetic | |
|---|---|
| variation type | Uniform resource location |
| SNP | http://bioinfo.pal.roche.com/usuka_bioinformatics/cgi- |
| | bin/msnp/msnp.pl |
| SNP | http://snp.cshl.org/ |
| SNP | http://www.ibc.wustl.edu/SNP/ |
| SNP | http://www-genome.wi.mit.edu/SNP/mouse/ |
| SNP | http://www.ncbi.nlm.nih.gov/SNP/ |
| Microsatellite | http://www.informatics.jax.org/searches/ |
| markers | polymorphism_form.shtml |
| Restriction | http://www.informatics.jax.org/searches/ |
| fragment | polymorphism_form.shtml |
| length | |
| polymorphisms | |
| Short tandem | http://www.cidr.jhmi.edu/mouse/mmset.html |
| repeats | |
| Sequence | http://mcbio.med.buffalo.edu/mit.html |
| length | |
| polymorphisms | |
| DNA | http://genome.imb-jena.de/public.html |
| methylation | |
| database | |

### 5.3 Construction of Haplotype Blocks

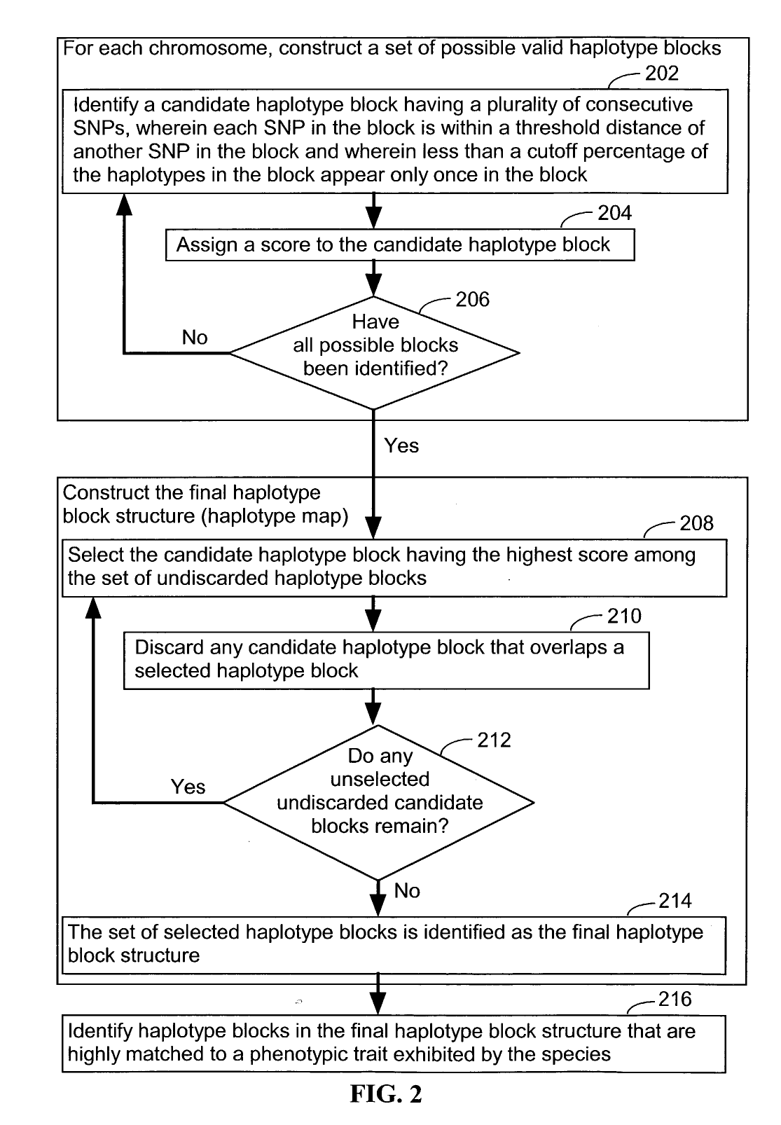

FIG. 2 illustrates a method that is performed in accordance with one embodiment of the present invention. The first several steps of the method illustrated in FIG. 2 are performed by haplotype map derivation subroutine 48 (FIG. 1) and result in the generation of a haplotype map that comprises haplotype blocks. These steps can be used in instances where genotypic database 52 includes SNP information. Genotypic database 52 is used as the input to haplotype map derivation subroutine 48. In other words, haplotype map derivation subroutine 48 generates haplotype blocks using the data in genotypic database 52.

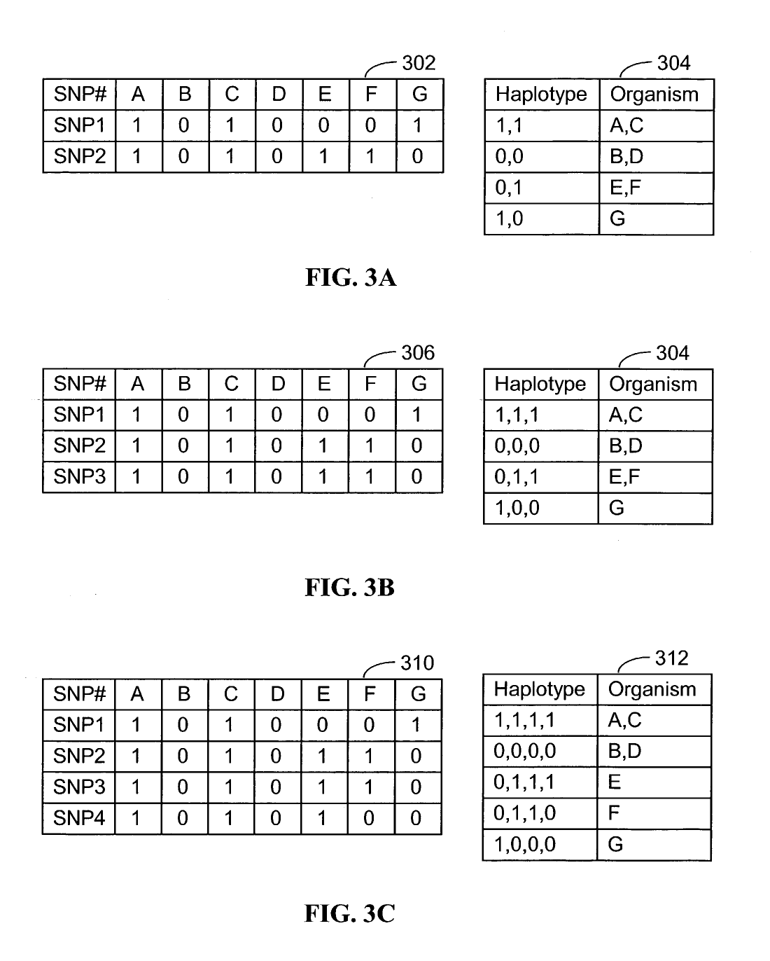

Before the steps illustrated in FIG. 2 are described in detail, a brief description of haplotype blocks is instructive. Generally speaking, a haplotype block represents a plurality of consecutive SNPs or other genetic variations (e.g., RFLPs, microsatellite markers, short tandem repeats, sequence length polymorphisms, or DNA methylation) in the genome of a species across a plurality of organisms in the species. Table 302 in FIG. 3A illustrates a haplotype block. In FIG. 3A, there are two SNPs (SNP1 and SNP2) that are adjacent to each other in the genome of a single species. The single species is represented by organisms A through G. Each organism has one value for each of SNP1 and SNP2, a major value “1” or a minor value “0”. Each value indicates whether the nucleotide at the locus represented by the SNP is more commonly found (major value, “1”) or less commonly found (minor value, “0”) at that locus in organisms of the species.

The respective nucleotides at the loci represented by SNP1 and SNP2 in organism A in FIG. 3A are nucleotides that are more commonly found in these loci. Accordingly, both SNP1 and SNP2 have a major value in organism A. In contrast, respective nucleotides at the loci represented by SNP1 and SNP2 in organism B in FIG. 3A are nucleotides that are less commonly found at these loci. Therefore, both SNP1 and SNP2 have aminor value in organism B.

In FIG. 3, organisms A and B have different haplotypes. In one embodiment, a haplotype is the collection of SNP values for a given organism in a given haplotype block. For example, a haplotype is the values in any of the columns representing an organism in FIG. 3. Organism A has a haplotype of 1,1 in FIG. 3A. Organism B has a haplotype of 0,0 in FIG. 3A. Table 304 lists all the haplotypes represented in table 302 in FIG. 3A as well as which organisms in the species have these haplotypes.

Now that the terms haplotype block and haplotype have been introduced, the method illustrated in FIG. 2 is described. In step 202, a candidate haplotype block having a plurality of consecutive SNPs in the genome of the single species under study is identified. To do this, haplotype map derivation routine 48 starts with the first SNP available to it and proceeds to build a haplotype block by adding to the block consecutive additional SNPs provided (1) the SNPs are within a threshold distance of the preceding SNP in the block and (2) no more than a predetermined threshold percentage of the haplotypes appear only once in the haplotype block. Whenever either of the above two conditions cannot be satisfied by the addition of the next consecutive SNP to the block then being formed, formation of the block is terminated. In some embodiments, (not shown) there is no requirement that the SNPs be within a threshold distance of the preceding SNP in the block. Upon terminating formation of the block at step 204, the haplotype map derivation routine 48 assigns a score to the haplotype block (step 206).

In various embodiments, the threshold distance between SNPs in a haplotype block is less than 10 megabases, less than 5 megabases, less than 3 megabases, less than 2 megabases, or less than 1 megabase. In some embodiments, there is no threshold distance requirement. In some embodiments, the predetermined threshold percentage of unique haplotypes in a haplotype block is within a range between 5 and 10, 10 and 15, 15 and 20, 20 and 25, 5 and 30, 15 and 25, 25 and 30, 30 and 40, or greater than 40.

FIG. 3 illustrates the application of the predetermined threshold percentage as applied in step 202. In FIG. 3A, there are four haplotypes in candidate haplotype block 302. Three of the haplotypes [(1,1), (0,0), and (0,1)] are each represented by two organisms used to construct the candidate haplotype block. Therefore, each of these haplotypes appears more than once in the haplotype block. The fourth haplotype (1,0) is only represented by a single organism. Thus, the fourth haplotype only appears once in the candidate haplotype block; and fully twenty-five percent of the haplotypes in haplotype block 302 are only represented by a single organism used to construct the candidate haplotype block. If the threshold percentage in step 202 is set at 20, then block 302 would not qualify as a candidate haplotype block. On the other hand, if the threshold percentage is set at 30, then block 302 would qualify as a candidate haplotype block. In a preferred embodiment, the threshold percentage is set at 20 and block 302 does not qualify as a candidate haplotype block. In FIG. 3B, there are three haplotypes that appear more than once in haplotype block 306 [(1,1,1), (0,0,0), (0,1,1)] and a single haplotype that appears only once (1,0,0). In FIG. 3C, there are only two haplotypes that appear more than once in haplotype block 310 [(1,1,1,1), (0,0,0,0)] while the remaining haplotypes only appear once in block 310. Thus, if the threshold percentage is set at 20, neither block 306 nor block 310 qualifies as a haplotype block; but, if the threshold percentage is set at 30, block 306 does qualify.

FIG. 3 illustrates another point relating to candidate haplotype blocks. There is no limit to the number of SNPs in a candidate haplotype block as long as the criteria imposed by step 202 are satisfied. In other words, there is no limit to the number of SNPs in a candidate haplotype block as long as (i) the SNPs in the block are consecutive, (ii) each SNP is within a cutoff distance of another SNP in the genome of the organism, and (iii) no more than a cutoff percentage of the haplotypes in the block are unique.

As noted above, after a candidate haplotype block is identified, it is assigned a score at step 204. In one embodiment of the present invention, this score is the number of SNPs within the block divided by the square of the number of different haplotypes in the block. To illustrate, candidate haplotype block 302 (FIG. 3A) has a score of 2 divided by four squared (0.125). Candidate haplotype block 306 (FIG. 3B) has a score of 3 divided by four squared (0.188). Candidate haplotype block 310 (FIG. 3C) has a score of 4 divided by five squared (0.160). Those of skill in the art will appreciate that there are a number of different scoring mechanisms that could be used to score candidate haplotype blocks and all such scoring mechanisms are within the scope of the present invention. For instance, in some embodiments, the scoring function used in step 204 is the number of SNPs within the block divided by the number of different haplotypes in the block. In other embodiments, the scoring function used in step 204 is the number of SNPs within the block divided by the number of different haplotypes in the block raised to a power greater than 2 (e.g., to the third power).

In step 206, a determination is made as to whether all possible candidate haplotype blocks have been generated from genotypic database 52. There are any number of methods by which this determination can be made. In one embodiment, all possible candidate haplotype blocks have been generated (206-Yes) from genotypic database 52 if there is no SNP remaining in database 52 that has not been considered for initiating formation of a new haplotype block. If not all possible blocks have been generated (206-No), control returns to step 202 and an attempt to identify another candidate haplotype block is initiated.

Once all possible candidate haplotype blocks in genotypic database 52 have been identified (206-Yes), the final haplotype block structure (haplotype map) is generated. Initially, all candidate haplotype blocks identified in instances of step 202 are eligible for consideration. In step 208, a candidate haplotype block having the highest score in the set of eligible candidate haplotype blocks is selected from the final haplotype block and is removed from the set of eligible candidate haplotype blocks. In step 210, any haplotype block that overlaps the haplotype block selected in step 208 is removed from the set of eligible candidate blocks, and thereafter ignored. Two haplotype blocks overlap each other when the two blocks share at least one common SNP. At this stage, it is possible to have overlapping haplotype blocks in the set of eligible haplotype blocks because steps 202 through 206 are designed to generate all possible qualified haplotype blocks, regardless of whether the blocks overlap each other.

In step 212, a determination is made as to whether any haplotype blocks remain in the set of eligible haplotype blocks. If so (212-Yes), control passes back to step 208 and the candidate haplotype block having the highest score among the set of remaining eligible candidate blocks is selected for inclusion in the final haplotype block. Steps 208 through 212 are repeated until no haplotypes blocks remain in the set of eligible haplotype blocks. The haplotype blocks that were selected in iterations of step 208 are identified as the final haplotype block (haplotype map) structure.

Steps 202 through 214 illustrate one method for deriving a haplotype block map. Steps 202 through 214 are useful for species in which small numbers of inbred strains (organisms) are studied and for which SNP data is available. However, the present invention is not limited to the haplotype block map constructions steps outlined in steps 202 through 214 of FIG. 2. Indeed, a haplotype block map produced using a variety of methods can be used in the methods of the present invention. For example, in instances where the species under study is human and there are a large number of organisms represented in genotypic database 52, methods such as those described in Patil et al., 2001, Science 294, 1719-1723; Daly et al., 2001, Nature Genetics 29, 229-232; and Zhang et al., 2002, Proceedings of the National Academy of Sciences of the United States of America 99, 7335-7339 can be used. Furthermore, the present invention is not limited to the construction of haplotype blocks based on SNPs. Any form of genetic variation can be used go generate haplotype blocks using methods similar to those described herein. Haplotype blocks can be constructed from genetic variations such as restriction fragment length polymorphisms (RFLPs), microsatellite markers, short tandem repeats, sequence length polymorphisms, and DNA methylation, to name a few. For example, Kong et al. describes techniques for the generation of a human haplotype map using microsatellite markers. See Kong et al., 2002, Nat. Genet 31, 241-247.

### 5.4 Empirical Mapping of Haplotype Blocks to Phenotypic Data

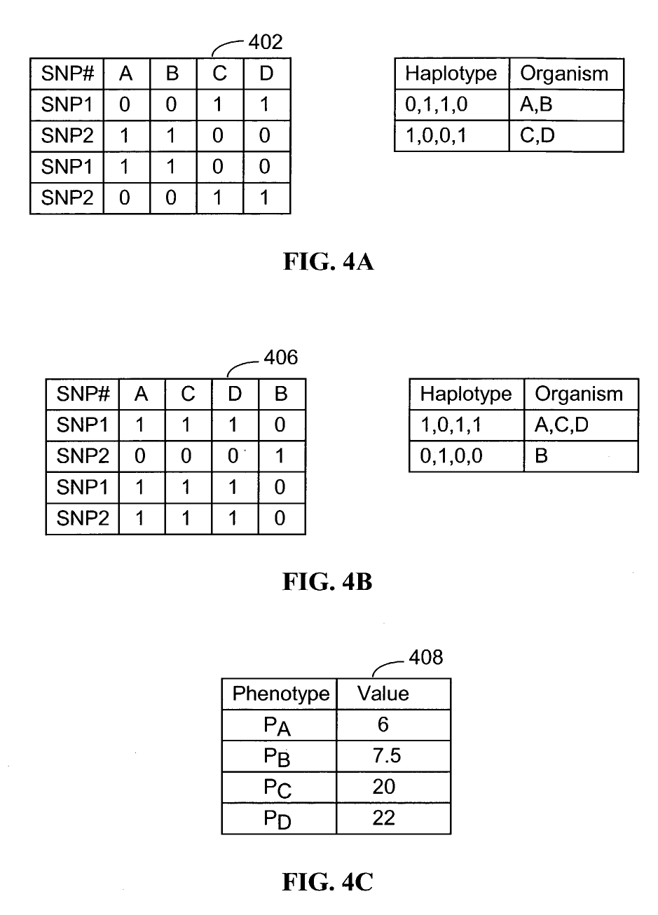

In step 216, the haplotype blocks in the final haplotype block structure that are most highly matched to a phenotypic trait exhibited by the species are identified. This is done by scoring each of the haplotype blocks in the final haplotype block structure against a phenotypic trait exhibited by the species under study. A scoring function used in step 216 in one embodiment of the present invention is illustrated using the hypothetical phenotypic data illustrated in FIG. 4. In this embodiment, a lower score indicates a better match between a phenotype and a haplotype block. The scoring function evaluates how well the distribution of alleles within a haplotype block match the hypothetical phenotypic data. As used herein, a better score produced by the scoring function used in step 216 is any score that represents a better match between a phenotype and a haplotype block. In some forms of scoring functions used in some embodiments of step 216, a better score is a lower score while in other forms of scoring functions used in some embodiments of step 216, a better score is a higher score.

FIG. 4 illustrates candidate haplotype blocks 402 and 404. Block 404 includes haplotype (0,1,1,0) which is represented by organisms A and B as well as haplotype (1,0,0,1) which is represented by organisms C and D. Block 406 includes haplotype (1,0,1,1) which is represented by organisms A, C, and D as well as haplotype (1,0,0,1) which is represented by organism B.

FIG. 4C illustrates values of hypothetical phenotypic data against which candidate haplotype blocks 402 and 404 are scored. The hypothetical phenotypic data could represent some phenotype of the species under study, such as, for example, lung capacity, blood cholesterol level, etc. There is a phenotypic value for each of the organisms represented by the candidate haplotype blocks. Thus organism A exhibits a phenotype PA having 6 arbitrary units, organism B exhibits a phenotype PB having 7.5 arbitrary units and so forth.

In this exemplary embodiment, the scoring function used in step 216 (FIG. 2) is:

$$S = - log ( \frac{\sum {D}_{intra}}{\sum \vert {D}_{inter} \vert} ) \qquad Eqn . 1$$

where,

Σᵢₙₜᵣₐ is the summation of the differences in phenotypic values for organisms that share the same haplotype in a haplotype block, and

ΣDᵢₙₜₑᵣ is the summation of the differences in phenotypic values between organisms that do not share the same haplotype in a haplotype block.

Equation 1 is the negative log of the ratio of the phenotypic difference within haplotype groups relative to the average phenotypic difference between haplotype groups.

To illustrate the computation of equation 1 for blocks 402 and 404, consider the complete set of differences in phenotypic values for set 408 (FIG. 4C):

D_{AB}=1.5

D_{AC}=14

D_{AD}=16

D_{BC}=12.5

D_{BD}=14.5

D_{CD}=2

The score S₄₀₂ for candidate haplotype blocks 402 is computed by considering that there are two haplotypes (0,1,1,0) and (1,0,0,1). Organisms A and B belong to one haplotype and organisms C and D belong to the other haplotype.

$$\begin{matrix}{S}_{402} = - log ( \frac{{D}_{AB} + {D}_{CD}}{\vert {D}_{\overline{AB}} - {D}_{\overline{CD}} \vert} ) \\ {S}_{402} = - log ( \frac{1.5 + 2}{21 - 6.75} )\end{matrix}$$

S₄₀₂=0.610

The score S₄₀₆ for candidate haplotype blocks 406 is computed by considering that there are two haplotypes (1,0,1,1) and (0,1,0,0). Organisms A, C, and D belong to one haplotype and organism B belongs to the other haplotype:

$$\begin{matrix}{S}_{406} = - log ( \frac{{D}_{AC} + {D}_{AD} + {D}_{CD}}{{D}_{\overline{ACD}} - {D}_{\overline{B}}} ) \\ {S}_{406} = - log ( \frac{14 + 16 + 2}{16 - 7.5} )\end{matrix}$$

S₄₀₆=−0.576

The scoring function set forth in Equation 1 indicates that block 402 is a better match against the hypothetical phenotypic data in FIG. 4C than block 406. Equation 1 is designed so that haplotype blocks in a haplotype block map that better match a phenotype exhibited by a single species receive a more positive score than haplotype blocks that do not match the phenotype.

5.4.1 Alternative Scoring Functions

Other scoring functions other than the one provided by Equation 1 may be used to score each haplotype block in a haplotype block map. In one embodiment, the scoring function is

$$S = ( \frac{\sum {D}_{intra}}{\sum \vert {D}_{inter} \vert} ) \qquad Eqn . 2$$

where, ΣDᵢₙₜᵣₐ and ΣDᵢₙₜₑᵣ have the same meaning as in Equation 1. Equation 2 emphasizes an advantage of the present invention. Equation 2 is capable of differentiating haplotype blocks in a haplotype map based on how well the haplotype blocks compare to phenotypic data for organisms represented in the haplotype blocks. As written, Equation 2 will assign a smaller number to haplotypes blocks that better match phenotypic data and a larger number to haplotypes that poorly match the phenotypic data. Equation 2.0 could just as easily be rewritten

$$S = - ( \frac{\sum {D}_{intra}}{\sum \vert {D}_{inter} \vert} ) \qquad Eqn . 3$$

where, ΣDᵢₙₜᵣₐ and ΣDᵢₙₜₑᵣ have the same meaning as in Eqn. 1. In the case of Equation 3, less negative numbers will be assigned to haplotypes blocks that better match phenotypic data and a more negative numbers will be assigned to haplotypes that poorly match the phenotypic data 3. The point is that the scoring function differentiates between haplotype blocks that more closely match a given phenotype from those haplotype blocks that less closely match a given phenotype.

Those of skill in the art will appreciate that there are a number of different scoring functions that can be used in step 216. In one embodiment, the scoring function is any function that differentiates between haplotype blocks that closely match a phenotype exhibited by the single species under study and haplotype blocks that do not closely match the phenotype. In other embodiments, the scoring function is any of Equations 1, 2 or 3, the negative of Equations 1, 2, or 3, the inverse of Equations 1, 2, or 3, or the inverse negative of Equations 1, 2, or 3. In still other embodiments, the scoring function is a logarithm of the ratio in Equation 2, a logarithm of the inverse ratio in Equation 2, or some other function of the ratio in Equation 2.

5.4.2 Weighted Scoring Functions

In some embodiments of the present invention, a weight is introduced into the numerator and/or the denominator of the ratio present in the scoring function. In some instances, this weight is a constant value. In other instances, the magnitude of the weight is a function of the number of organisms represented in the haplotype block being compared to the phenotypic data, a function of the number of SNPs (or other forms of genetic variations such as RFLPs) in the haplotype block being considered, or some other relevant aspect related to the underlying data. In some embodiments, the score is multiplied by a weight factor. For example, in some embodiments, the negative log ratio of Equation 1 is multiplied by a weight factor that reflects the size and structure of the haplotype block being scored.

In some embodiments of the present invention, the numerator and/or the denominator of the ratio present in the scoring function used in step 216 is raised to a power (e.g., the square root, square, or power of 10). For example, in some embodiments, the scoring function is

$$S = ( \frac{\sum \sqrt{{D}_{intra}}}{\sum \vert {D}_{inter} \vert} ) \qquad Eqn . 4$$

A number of different scoring functions that can be used in various embodiments of step 216 have been disclosed. These examples are by way of illustration only and not limitation. The techniques of the present invention are advantageous because they allow for the localization of genetic elements that affect phenotypes of a species to specific regions of the genome of a species. Analysis of the specific regions of the genome identified by the techniques of the present invention can then be analyzed further to identify specific genes that affect specific phenotypes exhibited by the species.

In some embodiments of the present invention, Equation 1 is used to score each of the haplotype blocks. Each score is multiplied by a weight that reflects the size and structure of the haplotype block being scored to yield a raw matching score. The raw matching score is normalized by subtracting away the mean raw score and dividing the standard deviation for all the haplotype blocks that are scored. The resulting scaled score indicates the number of standard deviations of score above or below the mean score.

### 5.5 Phenotypes

In some embodiments of the present invention, the techniques disclosed above are used to associate a phenotype exhibited by the species under study with specific haplotype blocks in the chromosome. In some embodiments, therefore, the methods of the present invention associate a phenotype exhibited by the species under study with a region of the chromosome that is less than 0.5 of a megabase (Mb), less than 1 Mb, less than 2 Mb, between 0.5 Mb and 2 Mb, less than 3 Mb, less than 4 Mb, between 2 Mb and 5 Mb, less than 5 Mb, less than 10 Mb, between 1 Mb and 10 Mb, less than 15 Mb, or less than 20 Mb.

The phenotypes that can be analyzed using the present invention are any form of complex trait (as opposed to a simple Mendelian trait). A complex trait includes any trait that can be measured on a continuum. So, for example, a complex trait can be height, weight, levels of biological molecules in the blood, and susceptibility to a disease, to name a few. In some embodiments, the complex trait that is studied is a complex disease such as diabetes, cancer, asthma, schizophrenia, arthritis, multiple sclerosis, and rheumatosis. In some embodiments, the phenotype that is studied is a preclinical indicator of disease, such as, but not limited to, high blood pressure, abnormal triglyceride levels, abnormal cholesterol levels, or abnormal high-density lipoprotein/low-density lipoprotein levels. In a specific embodiment of the present invention, the phenotype is low resistance to an infection by a particular insect or pathogen. Additional exemplary phenotypes that may be studied using the systems and methods of the present invention include allergies, asthma, and obsessive-compulsive disorders, such as panic disorders, phobias, and post-traumatic stress disorders.

Still other phenotypes that may be studied using the methods of the present invention include diseases such as autoimmune disorders (e.g., Addison's disease, alopecia areata, ankylosing spondylitis, antiphospholipid syndrome, Behcet's disease, chronic fatigue syndrome, Crohn's disease and ulcerative colitis, diabetes, fibromyalgia, Goodpasture syndrome, graft versus host disease, lupus, Meniere's disease, multiple sclerosis, myasthenia gravis, myositis, pemphigus vulgaris, primary biliary cirrhosis, psoriasis, rheumatic fever, sarcoidosis, scleroderma, vasculitis, vitiligo, and Wegener's granulomatosis) bone diseases (e.g., achondroplasia, bone cancer, fibrodysplasia ossificans progressiva, fibrous dysplasia, legg calve perthes disease, myeloma, osteogenesis imperfecta, osteomyelitis, osteoporosis, paget's disease, and scoliosis.

Still other phenotypes that may be studied using the methods of the present invention include cancers such as bladder cancer, bone cancer, brain tumors, breast cancer, cervical cancer, colon cancer, gynecologic cancers, Hodgkin's disease, kidney cancer, laryngeal cancer, leukemia, liver cancer, lung cancer, lymphoma, oral cancer, ovarian cancer, pancreatic cancer, prostate cancer, skin cancer, and testicular cancer.

Still other phenotypes that may be studied using the methods of the present invention include genetic disorders such as achondroplasia, achromatopsia, acid maltase deficiency, adrenoleukodystrophy, Aicardi syndrome, alpha-1 antitrypsin deficiency, androgen insensitivity syndrome, Apert syndrome, dysplasia, ataxia telangiectasia, blue rubber bleb nevus syndrome, canavan disease, Cri du chat syndrome, cystic fibrosis, Dercum's disease, fanconi anemia, fibrodysplasia ossificans progressiva, fragile x syndrome, galactosemia, gaucher disease, hemochromatosis, hemophilia, Huntington's disease, Hurler syndrome, hypophosphatasia, klinefelter syndrome, Krabbes disease, Langer-Giedion syndrome, leukodystrophy, long qt syndrome, Marfan syndrome, Moebius syndrome, mucopolysaccharidosis (mps), nail patella syndrome, nephrogenic, diabetes insipidus, neurofibromatosis, Niemann-Pick disease, osteogenesis imperfecta, porphyria, Prader-Willi syndrome, progeria, proteus syndrome, retinoblastoma, Rett syndrome, rubinstein-taybi syndrome, Sanfilippo syndrome, Shwachman syndrome, sickle cell disease, Smith-Magenis syndrome, Stickler syndrome, Tay-Sachs, thrombocytopenia absent radius (tar) syndrome, Treacher collins syndrome, trisomy, tuberous sclerosis, Turner's syndrome, urea cycle disorder, Von Hippel-Lindau disease, Waardenburg syndrome, Williams syndrome, and Wilson's disease.

Still other phenotypes that may be studied using the systems and methods of the present invention include angina pectoris, dysplasia, atherosclerosis/arteriosclerosis, congenital heart disease, endocarditis, high cholesterol, hypertension, long qt syndrome, mitral valve prolapse, postural orthostatic tachycardia syndrome, and thrombosis.

Yet other phenotypes that may be studied using the systems and methods of the present invention include the life-span of the organisms, the basal serum level of an antibody in the blood of the organisms, the serum level of an antibody in the blood of the organisms after exposure of the organism to a perturbation, the response of an organism in a pain model after the organism has been exposed to a pain relieving drug, etc.

### 5.6 Exemplary Phenotypic Data

In some embodiments of the present invention, phenotypic data structure 60 is microarray expression data. Microarrays are capable of quantitatively measuring the level of expression of thousands of genes; making it feasible to generate large databases of strain and tissue-specific gene expression data. See, for example, Zhao et al., 1995, “High-density cDNA filter analysis: a novel approach for large-scale, quantitative analysis of gene expression,” Gene 156: 207-213; Blanchard et al., 1996, “Sequence to array: Probing the genome's secrets,” Nature Biotechnology 14:1649; Blanchard et al., 1996, “High-Density Oligonucleotide Arrays,” Biosensors & Bioelectronics 11:687-90; Chee et al., 1996, “Accessing Genetic Information with High-Density DNA Arrays,” Science 274:610-614; Chait, 1996, “Trawling for proteins in the post-genome era,” Nat. Biotech. 14:1544; DeRisi et al., 1996, “Use of a cDNA microarray to analyze gene expression patterns in human cancer,” Nature Genetics 14:457-460; and DeRisi et al., 1997, “Exploring the metabolic and genetic control of gene expression on a genomic scale,” Science 278:680-686; Schena et al., 1995, “Quantitative monitoring of gene expression patterns with a complementary DNA micro-array,” Science 270: 467-470; Schena et al., 1996, “Parallel human genome analysis; microarray-based expression monitoring of 1000 genes,” Proc. Natl. Acad. Sci. USA 93:10614-10619; Shalon et al., 1996, “A DNA microarray system for analyzing complex DNA samples using two-color fluorescent probe hybridization,” Genome Res. 6:639-645.

In some embodiments of the present invention, the average expression level for a gene or gene products on the microarray is used as input, and variation in the data is used as a weighting factor. This capability allows for more accurate computational mapping of strain-specific gene expression data onto haplotype blocks. See, for example, Use Case 3 in Example 2, below.

5.6.1 Microarrays Generally

In a some embodiments of the present invention, phenotypic data structure 60 includes measurements of the transcriptional state of organisms 56 of a single species. In some embodiments transcriptional state measurements are made by hybridizing probes to microarrays consisting of a solid phase. On the surface of the solid phase are a population of immobilized polynucleotides, such as a population of DNA or DNA mimics, or, alternatively, a population of RNA. Microarrays can be employed, e.g., for analyzing the transcriptional state of a cell, such as the transcriptional states of cells exposed to graded levels of a drug of interest.

In some embodiments, a microarray comprises a surface with an ordered array of binding (e.g., hybridization) sites for products of many of the genes in the genome of a cell or organism, preferably most or almost all of the genes. Microarrays can be made in a number of ways, of which several are described below. However produced, microarrays share certain characteristics: the arrays are reproducible, allowing multiple copies of a given array to be produced and easily compared with each other. Preferably, the microarrays are small, usually smaller than 5 cm², and they are made from materials that are stable under binding (e.g., nucleic acid hybridization) conditions. Preferably, a given binding site or unique set of binding sites in the microarray will specifically bind (e.g., hybridize) to the product of a single gene in a cell (e.g., to a specific mRNA, or to a specific cDNA derived therefrom). However, in general, other, related or similar sequences will cross-hybridize to a given binding site. Although there may be more than one physical binding site per specific RNA or DNA, for the sake of clarity the discussion below will assume that there is a single, completely complementary binding site.

The microarrays in accordance with one embodiment of the present invention include one or more test probes, each of which has a polynucleotide sequence that is complementary to a subsequence of RNA or DNA to be detected. Each probe preferably has a different nucleic acid sequence. The position of each probe on the solid surface is preferably known. In one embodiment, the microarray is a high density array, preferably having a density greater than about 60 different probes per 1 cm². In one embodiment, the microarray is an array (e.g., a matrix) in which each position represents a discrete binding site for a product encoded by a gene (e.g., an mRNA or a cDNA derived therefrom), and in which binding sites are present for products of most or almost all of the genes in the genome of the species. For example, the binding site can be a DNA or DNA analogue to which a particular RNA can specifically hybridize. The DNA or DNA analogue can be, e.g., a synthetic oligomer, a full-length cDNA, a less-than full length cDNA, or a gene fragment.

Although in some embodiments the microarray contains binding sites for products of all or almost all genes in the genome of the single species, such comprehensiveness is not necessarily required. In some instance, the microarray will have binding sites corresponding to at least 50%, at least 75%, at least 85%, at least 90%, or at least 99% of the genes in the genome. Preferably, the microarray has binding sites for genes relevant to the action of a drug of interest or in a biological pathway of interest. A “gene” is identified as an open reading frame (“ORF”) that encodes a sequence of preferably at least 50, 75, or 99 amino acids from which a messenger RNA is transcribed in the organism or in some cell in a multicellular organism. The number of genes in a genome can be estimated from the number of mRNAs expressed by the organism, or by extrapolation from a well characterized portion of the genome. When the genome of the organism of interest has been sequenced, the number of ORF's can be determined and mRNA coding regions identified by analysis of the DNA sequence. For example, the genome of Saccharomyces cerevisiae has been completely sequenced, and is reported to have approximately 6275 ORFs longer than 99 amino acids. Analysis of the ORFs indicates that there are 5885 ORFs that are likely to encode protein products (Goffeau et al., 1996, Science 274:546-567).

5.6.2 Preparing Probes for Microarrays

As noted above, the “probe” to which a particular polynucleotide molecule specifically hybridizes in some embodiment of the invention is a complementary polynucleotide sequence. In one embodiment, the probes of the microarray are DNA or DNA “mimics” (e.g., derivatives and analogues) corresponding to at least a portion of each gene in the genome of a species. In some embodiments, the probes of the microarray are complementary RNA or RNA mimics.

DNA mimics are polymers composed of subunits capable of specific, Watson-Crick-like hybridization with DNA, or of specific hybridization with RNA. The nucleic acids can be modified at the base moiety, at the sugar moiety, or at the phosphate backbone. Exemplary DNA mimics include, e.g., phosphorothioates.

DNA can be obtained, for example, by polymerase chain reaction (“PCR”) amplification of gene segments from genomic DNA, cDNA (e.g., by RT-PCR), or clones sequences. PCR primers are preferably chosen based on known sequences of the genes or cDNA that result in amplification of unique fragments (e.g, fragments that do not share more than 10 bases of contiguous identical sequence with any other fragment on the microarray). Computer programs that are well known in the art are useful in the design of primer with the required specificity and optimal amplification properties, such as Oligo version 5.0 (National Biosciences). Typically, each probe of the microarray will be between about 20 bases and about 12,000 bases, and usually between about 300 bases and about 2,000 bases in length, and still more usually between about 300 bases and about 800 bases in length. PCR methods are well known in the art, and are described, for example, in Innis et al., eds., 1990, PCR Protocols: A Guide to Methods and Applications, Academic Press Inc., San Diego, Calif.

An alternative means for generating the polynucleotide probes of the microarray is by synthesis of synthetic polynucleotides or oligonucleotides, e.g., using N-phosphonate or phosphoramidite chemistries (Froehler et al., 1986, Nucleic Acid Res. 14:5399-5407; McBrid et al., 1983, Tetrahedron Lett. 24:246-248). Synthetic sequences are typically between about 15 and about 500 bases in length, more typically between about 20 and about 50 bases. In some embodiments, synthetic nucleic acids include non-natural bases, such as, but by no means limited to, inosine. As noted above, nucleic acid analogues may be used as binding sites for hybridization. An example of a suitable nucleic acid analogue is peptide nucleic acid (see, e.g., Egholm et al., 1993, Nature 363:566-568; U.S. Pat. No. 5,539,083).

In alternative embodiments, the hybridization sites (e.g., the probes) are made from plasmid or phage clones of genes, cDNAs (e.g., expressed sequence tags), or inserts therefrom (Nguyen et al., 1995, Genomics 29:207-209).

5.6.3 Attaching Probes to the Solid Surface of Microarrays

The probes are attached to a solid support or surface, which may be made, e.g., from glass, plastic (e.g., polypropylene, nylon), polyacrylamide, nitrocellulose, or other materials. A preferred method for attaching the nucleic acids to a surface is by printing on glass plates, as is described generally by Schena et al., 1995, Science 270:467-470. This method is especially useful for preparing microarrays of cDNA

A second preferred method for making microarrays is by making high-density oligonucleotide arrays. Techniques are known for producing arrays containing thousands of oligonucleotides complementary to defined sequences, at defined locations on a surface using photolithographic techniques for synthesis in situ (see, Fodor et al., 1991, Science 251:767-773; Lockhart et al., 1996, Nature Biotechnology 14:1675; U.S. Pat. Nos. 5,578,832; 5,556,752; and 5,510,270) or other methods for rapid synthesis and deposition of defined oligonucleotides (Blanchard et al., Biosensors & Bioelectronics 11:687-690). When these methods are used, oligonucleotides (e.g., 20-mers) of known sequence are synthesized directly on a surface such as a derivatized glass slide. Usually, the array produced is redundant, with several oligonucleotide molecules per RNA. Oligonucleotide probes can be chosen to detect alternatively spliced mRNAs.

Other methods for making microarrays, e.g., by masking (Maskos and Southern, 1992, Nuc. Acids. Res. 20:1679-1684), may also be used. In principle, any type of array, for example, dot blots on a nylon hybridization membrane could be used.

5.6.4 Other Sources of Phenotypic Data

The present invention provides additional sources of phenotypic data for phenotypic data structure 60 (FIG. 2). For example, in addition to the microarray techniques described above, the transcriptional state of a cell may be measured by gene expression technologies known in the art. Several such technologies produce pools of restriction fragments of limited complexity for electrophoretic analysis, such as methods combining double restriction enzyme digestion with phasing primers (see, e.g., European Patent O 534858 A1, filed Sep. 24, 1992, by Zabeau et al.), or methods selecting restriction fragments with sites closest to a defined mRNA end (see, e.g., Prashar et al., 1996, Proc. Natl. Acad. Sci. U.S.A. 93:659-663). Other methods statistically sample cDNA pools, such as by sequencing sufficient bases (e.g., 20-50 bases) in each of multiple cDNAs to identify each cDNA, or by sequencing short tags (e.g., 9-10 bases) which are generated at known positions relative to a defined mRNA end (see, e.g., Velculescu, 1995, Science 270:484-487).

In various embodiments of the present invention, aspects of the biological state other than the transcriptional state, such as the translational state, the activity state, or mixed aspects thereof can be measured in order to obtain phenotypic data for phenotypic data structure 60. Details of these embodiments are described in this section.

Translational State Measurements. Measurements of the translational state may be performed according to several methods. For example, whole genome monitoring of protein (e.g., the “proteome,” Goffea et al., supra) can be carried out by constructing a microarray in which binding sites comprise immobilized, preferably monoclonal, antibodies specific to a plurality of protein species encoded by the cell genome. Preferably, antibodies are present for a substantial fraction of the encoded proteins, or at least for those proteins relevant to the action of a drug of interest. Methods for making monoclonal antibodies are well known (see, e.g., Harlow and Lane, 1988, Antibodies: A Laboratory Manual, Cold Spring Harbor, N.Y.). With such an antibody array, proteins from the cell are contacted to the array, and their binding is assayed with assays known in the art.

Alternatively, proteins can be separated by two-dimensional gel electrophoresis systems. Two-dimensional gel electrophoresis is well known in the art, and typically involves iso-electric focusing along a first dimension followed by SDS-PAGE electrophoresis along a second dimension. See, e.g., Hames et al., 1990, Gel Electrophoresis of proteins: A Practical Approach, IRL Press, New York; Shevchenko et al., 1996, Proc. Natl. Acad. Sci. U.S.A. 93:1440-1445; Sagliocco et al., 1996, Yeast 12:1519-1533; and Lander, 1996, Science 274:536-539. The resulting electropherograms can be analyzed by numerous techniques, including mass spectrometric techniques, western blotting, and immunoblot analysis using polyclonal and monoclonal antibodies, and internal and N-terminal micro-sequencing. Using these techniques, it is possible to identify a substantial fraction of all the proteins produced under given physiological conditions, including in cells (e.g., in yeast) exposed to a drug, or in cells modified by, e.g., deletion or over-expression of a specific gene.

Activity State Measurements. In some embodiments of the present invention, phenotypic data used to construct phenotypic data structure 60 is activity state measurements of proteins in the organisms 56 of a single species. Activity measurements can be performed by any functional, biochemical, or physical means appropriate to the particular activity being characterized. Where the activity involves a chemical transformation, cellular protein can be contacted with the natural substrate(s), and the rate of transformation measured. Where the activity involves association in multimeric units, for example association of an activated DNA binding complex with DNA, the amount of associated protein or secondary consequences of the association, such as amounts of mRNA transcribed, can be measured. Also, where only a functional activity is known, for example, as in cell cycle (control, performance of the function can be observed. However known or measured, the changes in protein activities form the response data that can be matched with haplotype blocks using the methods of the present invention.

Mixed Aspects of Biological State. In alternative and non-limiting, embodiments, phenotypic data structure (FIG. 2) may be formed using mixed aspects of the biological state of cellular constituents (e.g., genes, proteins, mRNA, cDNA, etc.) within a plurality of different organisms of a single species. For example, response data can be constructed from combinations of, e.g., changes in certain mRNA abundance, changes in certain protein abundance, and changes in certain protein activities.

In addition to the examples provided in this Section, there are any number of sources of data that can be used to make quantitative measurements of complex traits. For example, the level of compounds in the blood can be analyzed, obesity measurement models can be used, etc.

### 5.7 Species and Organisms

The systems and methods of the present invention may be used to associate phenotypes with chromosomal locations in a variety of species. In some embodiments of the present invention, the species under study is an animal such as a mammal, primates, humans, rats, dogs, cats, chickens, horses, cows, pigs, mice, or monkeys. In yet other specific embodiments, the species under study is a plant, Drosophila, a yeast, a virus, or C. elegans. However, it is believed that the use of highly inbred organism (e.g., various mouse strains) will yield improved results. Each organism of the species is a member of the species (e.g. a particular mouse strain), a cellular tissue or organ derived from a member of the species (e.g., a mouse brain obtained from a particular mouse strain), or a cell culture derived from a member of the species.

### 5.8 Factors that Affect the Performance of the Computational Analysis

A number of factors affect the performance of the computational analysis. The methods of the present invention perform well when phenotypic data structure 60 (FIG. 1) reflects the genetic variation present within a haplotype block within genotypic database 52. A lack of information in either phenotypic data structure 60 or haplotypic information for some critical organisms 56 (strains) will adversely affect the performance of the empirical mapping. The number of organisms 56 analyzed is another important factor. The computational predictions are based upon the number of different organisms 56 compared. The number of pairwise comparisons is a combinatorial function of the number of strains analyzed. A haplotype map covering 40 to 50 commonly used inbred mouse strains would enable the computational prediction method of the present invention to have substantial power to identify genetic loci regulating a wide range of disease-associated phenotypic traits.

In some embodiments of the present invention, there is genotypic data for between 5 and 1000 organisms 56 in genotypic database 52. In some embodiments of the present invention, there are between 10 and 100 organisms 56 in genotypic database 52. In some embodiments of the present invention, there are between 20 and 75 organisms 56 in genotypic database 52.

### 5.9 Elucidating Biological Pathways

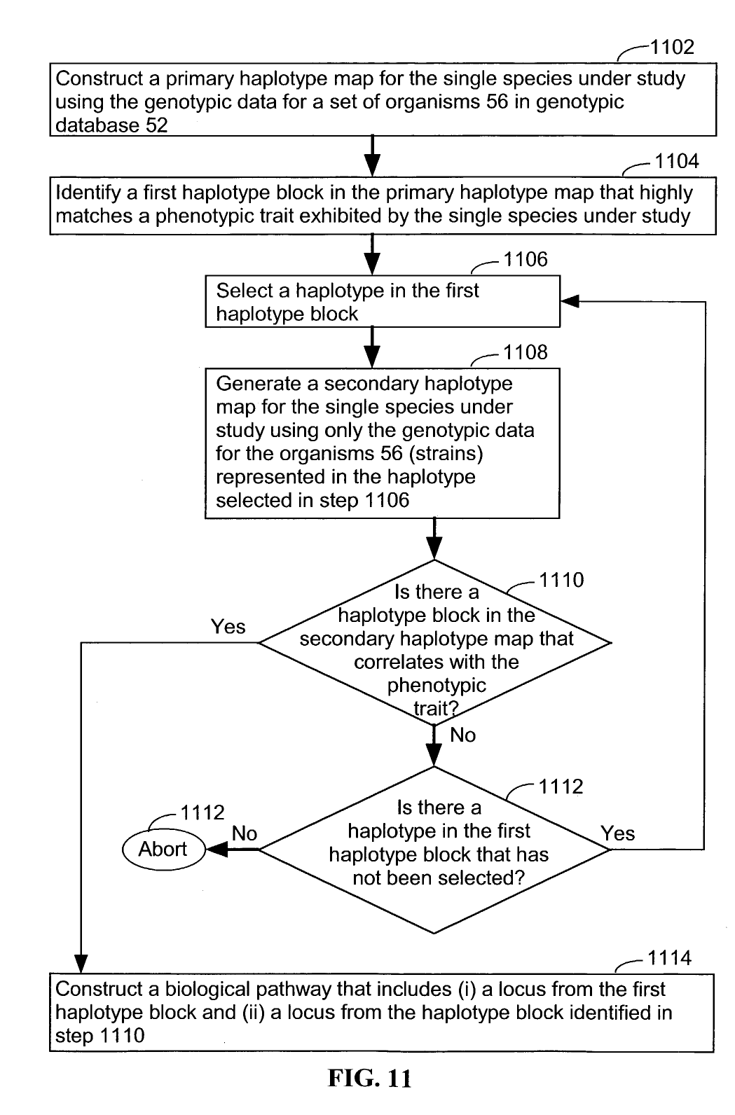

FIG. 11 illustrates a method for elucidating a biological pathway that exists in the single species under study using the systems and methods of the present invention. A biological pathway is used herein to mean any biological process in which a gene or gene product affects the expression or function of another gene or gene product in the species under study.

In step 1102, a primary haplotype map for the single species under study is constructed using the genotypic data for a set of organisms 56 in genotypic database 52. This can be done, for example, using steps 202 through 214 (FIG. 2). Next, in step 1104, a first haplotype block is identified in the primary haplotype map that highly matches a phenotypic trait exhibited by the single species under study. This can be done, for example, using the techniques described above in relation to step 216 of FIG. 2.

At this stage of the method, the haplotypes in the haplotype block identified in step 1104 are examined. Each haplotype in the block is represented by one or more organisms 56 in genotype database 52. In step 1106, a haplotype in the haplotype block identified in step 1104 is selected and, in step 1108, a secondary haplotype map is constructed using only that data 58 from the organisms 56 in database 52 (FIG. 2) that are in the haplotype identified in step 1106. Because only a subset of the organisms 56 are used to construct the secondary haplotype map, the haplotype blocks in the secondary haplotype map are likely to be different from those in the primary haplotype map. Construction of a secondary haplotype map is advantageous because it provides a method for subdividing a genotypic database 52 into subgroups. Analysis of these subgroups, in turn, can identify additional genes that affect a phenotype of interest in the species under study. The remaining steps in FIG. 11 provide one method in which these subgroups can be analyzed. However, one of skill in the art will appreciate that there are many modifications to the method comprising steps 1110 through 1120 of FIG. 11 and all such modifications are within the scope of the present invention.

In step 1110, a determination is made as to whether there is a haplotype block in the secondary haplotype map that correlates with the phenotypic trait. In the nontrivial case, this haplotype block in the secondary haplotype map will not overlap with the first haplotype block identified in step 1104. If a haplotype block in the secondary haplotype map that correlates with the phenotypic trait is found (1110-Yes), a biological pathway that includes (i) a locus from the first haplotype block, identified in step 1104, and (ii) a locus form the haplotype block identified in step 1110 is elucidated.

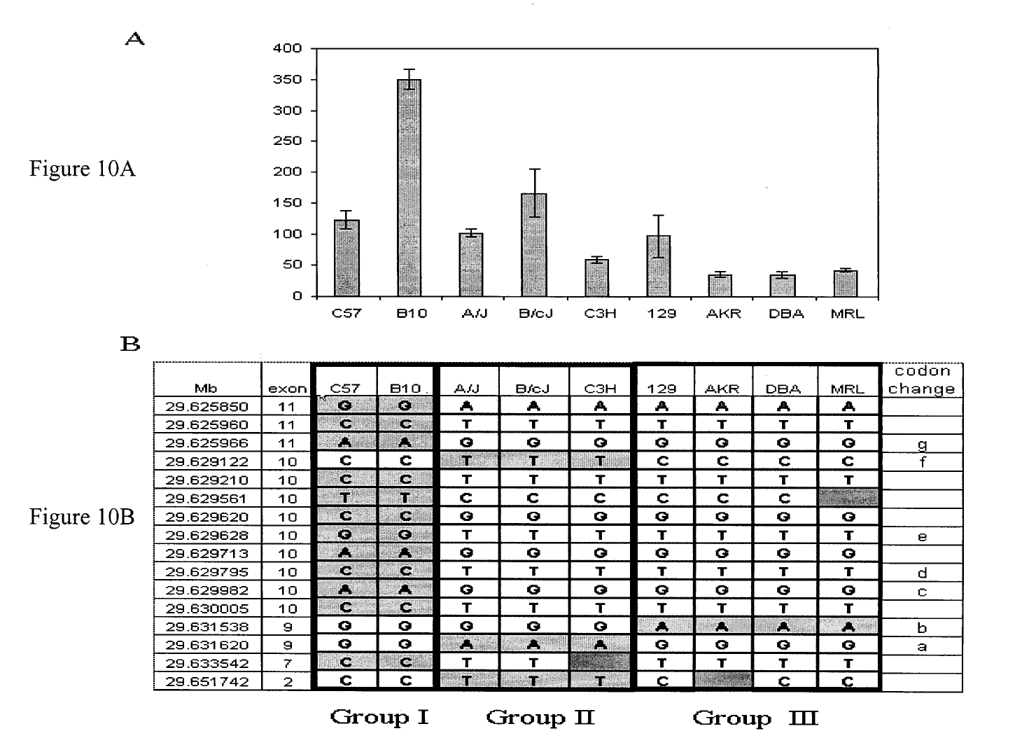

An example of the execution of step 1114 is found in Section 5.10.3 below. In Section 5.10.3, a haplotype block that correlates with Cyp1a1 expression in mice was identified (step 1104). As detailed in Section 5.10.3, this haplotype block includes a portion of the mouse genome that includes the aromatic hydrocarbon receptor (Ahr) locus. This haplotype block is illustrated in FIG. 10B. In Section 5.10.3, the species represented in Group III of the haplotype block illustrated in FIG. 10B were used to construct a secondary haplotype map (FIG. 11; step 1108). The secondary haplotype map included a haplotype block that correlates with Cyp1a1 expression (FIG. 11; step 1110-Yes). This secondary haplotype block included the Arnt locus. From this data, a determination was made that high expression of the Arnt gene product can modify the effect of the Ahr locus in mice as detailed in Section 5.10.3 (step 1114).

Returning to FIG. 11, in the case where a haplotype block is not found in the secondary map that correlates with the phenotypic trait under study, a determination is made as to whether any other unselected haplotypes remain in the first haplotype block (1112). If so, (1112-Yes), one such haplotype is selected 1106 and steps 1108 and 1110 are repeated. If not, (1112-No), the process aborts (1120).

### 5.10 EXAMPLES

In Example 1, the characteristics of haplotype blocks generated using the techniques disclosed in FIG. 2 as a function of the number of strains (organisms) present in genotypic database 52 are presented. In Example 2, the systems and methods of the present invention are used to correlate phenotypic data obtained from inbred mouse strains with haplotype blocks. In Example 3, the systems and methods of the present invention are used to construct a biological pathway. In Example 4, the systems and methods of the present invention are used to determine which chromosomal regions are responsive to a perturbation.

### 5.10.1 Example 1

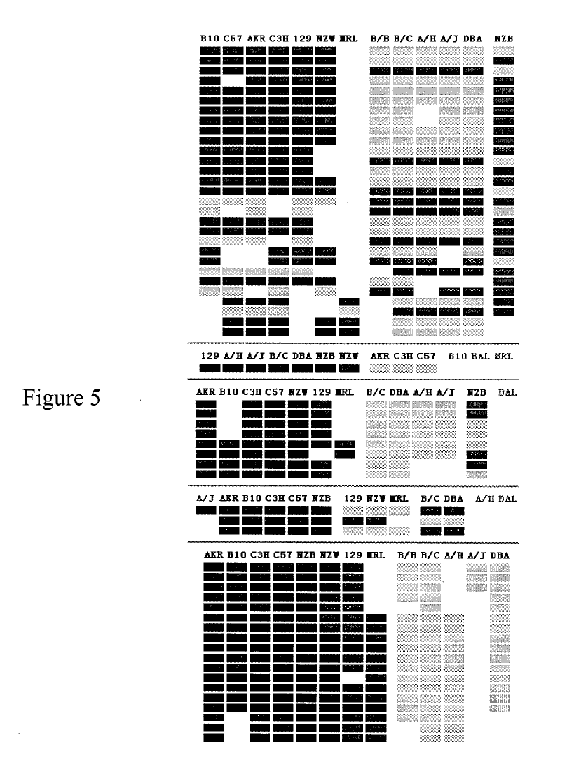

The exemplary genotypic database 52 used in this example is available at (http:\\mouseSNP.Roche.com). SNP discovery and allele characterization were performed using an automated, high-throughput method for re-sequencing of targeted genomic regions. See Grupe et al., 2001, Science 292, 1915-1918. The genomic regions analyzed were all within known biologically important genes; exons and key intra-genic regulatory regions within the genes were analyzed. The allelic information in exemplary genotypic database 52 was analyzed to characterize the pattern of genetic variation among these inbred mouse strains. As noted for SNPs in the human genome (see, for example, Patil et al., 2001, Science 294, 1719-1723; Daly et al., 2001, Nature Genetics 29, 229-232; Johnson et al., 2001, Nature Genetics 29, 233-237) alleles in close physical proximity in the mouse genome are often correlated, resulting in the presence of ‘SNP haplotypes’ appearing within block-like structures (FIG. 5). Each haplotype within a block apparently originates from a common ancestral chromosome; while the size of a block reflects other processes, including recombination and mutation.

There are several methods for defining a haplotype block, and the suitable definition depends on the anticipated application. For analyses of human genetic variation, the haplotype block structure is generated with the goal of minimizing the total number of SNPs required to cover a significant percentage of the haplotypic diversity within each block. See, for example, Patil et al., 2001, Science 294, 1719-1723; Daly et al., 2001, Nature Genetics 29, 229-232; and Zhang et al., 2002, Proceedings of the National Academy of Sciences of the United States of America 99, 7335-7339. This type of haplotype block structure is useful for human genetic analysis, which requires genotyping a large number of individuals for association studies. However, this approach does not produce an optimal block structure for experimental murine genetics; which involves characterization of a smaller number of inbred strains. More precise results are generated for association studies in mice by examining blocks that are smaller in size, and which have a less diverse haplotypic composition.

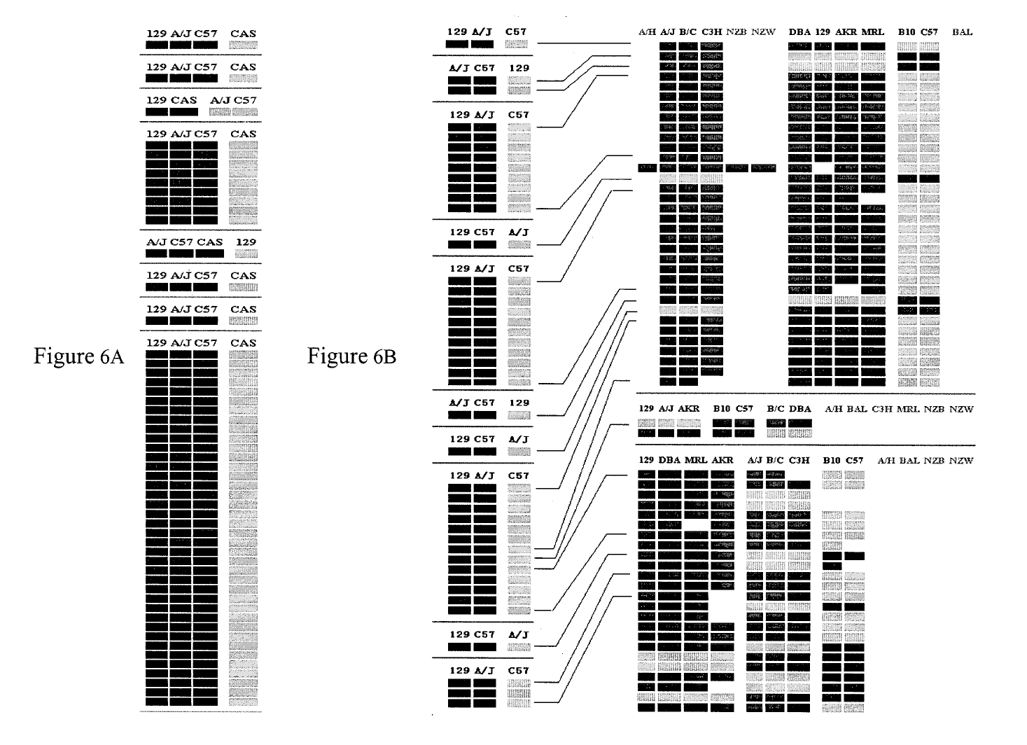

Because of the desire for haplotype blocks that have smaller size than those haplotype blocks generated using known methods, the novel method comprising steps 202 through 214 in FIG. 2 was used to analyze murine genetic variation and to define the haplotype block structure of the mouse genome. This method analyzes all SNPs (regardless of allele frequency) and all haplotypes (not just the common ones) for construction of haplotype blocks. Of importance, the number and type of strains included in the analysis significantly affected the structure of the haplotype blocks. As an example, the structure of haplotype blocks resulting from analysis of just 4 strains (129/SvJ, A/J, C57BL/6J and CAST/Ei) (FIG. 6A) was compared to that generated using 13 inbred Mus Musculus strains (not shown). Analysis of the genetic variation present in four strains generated a skewed haplotype block structure, as shown in the haplotype blocks on chromosome 1. In this situation, over 33% of the 94 haplotype blocks generated had CAST/Ei as the only strain with the minor allele (i.e. CAST/Ei had a unique haplotype not present in any other strain). For this reason, SNPs with only the CAST/Ei or SPRET/Ei strains having the minor allele were not used for haplotype block construction; and the haplotype blocks were based upon analysis of genetic variation among the 13 Mus Musculus strains. The general properties of the haplotype blocks on chromosome 1 generated by analysis of 13 Mus Musculus strains using steps 202 through 214 of FIG. 2 are shown in Table 2.

TABLE 2
Properties of the haplotype blocks on Mus Musculus chromosome 1

| | | | Avg. Num of | | Total |
|---|---|---|---|---|---|
| SNPs | Num of | Avg. size per | haplotype per | % of | block size |
| per block | blocks | block (Kb) | block | SNPs | (Mb) |
| >10 | 24 | 106 | 3.25 | 59 | 2.55 |
| 4-10 | 47 | 94 | 2.36 | 22 | 4.42 |
| 2-3 | 69 | 50 | 2.30 | 12 | 3.44 |
| 1 | 79 | N/A | 2 | 6 | N/A |
| Total | 219 | 74 | 2.31 | 100 | 10.41 |

Even when the analysis is confined to Mus Musculus strains, the number of strains analyzed significantly affected the structure of the haplotype blocks. When polymorphisms from an increasing number of Mus Musculus strains were analyzed; the number of SNPs increased as additional genetic variation was included in the analysis. The haplotype map constructed using only 3 strains was significantly different from that obtained using 13 strains (FIG. 6B). FIG. 6B is a comparison of haplotype blocks constructed on chromosome 12 (29.6 megabases) using 3 (A/J, 129 and C57BL/6) or 13 Mus Musculus strains. SNPs present at the boundary of blocks are joined by lines.

As the number of strains analyzed increased from 3 to 13, the general structure of the haplotype blocks stabilized as new strains were included in the analysis (Table 3).

TABLE 3
Properties of the haplotype blocks on Mus Musculus chromosome 1
as a function of the number of strains used in the computation

| | | | | Avg. | | | |
|---|---|---|---|---|---|---|---|
| | | Total | | no. | Avg. no. | % of | Max. |
| | Min. | No. | | of SNPs | of | SNPs | block |
| No. of | strain | of | No. of | per | haplotypes | in | length |
| Strains | No. | SNPs | blocks* | block* | per block* | block* | SNPs |
| 13 | 7 | 1270 | 71 | 14.61 | 2.66 | 82 | 108 |
| 12 | 7 | 1139 | 67 | 14.01 | 2.57 | 82 | 104 |
| 11 | 6 | 1248 | 68 | 15.41 | 2.62 | 84 | 106 |
| 10 | 6 | 1139 | 65 | 14.25 | 2.45 | 81 | 101 |
| 9 | 5 | 1225 | 66 | 15.33 | 2.48 | 83 | 104 |
| 8 | 5 | 1056 | 77 | 10.49 | 2.39 | 77 | 67 |
| 7 | 4 | 1228 | 96 | 9.27 | 2.21 | 72 | 81 |
| 6 | 4 | 1101 | 81 | 9.98 | 2.19 | 73 | 44 |
| 5 | 3 | 1067 | 75 | 10.99 | 2.11 | 77 | 80 |
| 4 | 3 | 933 | 72 | 8.74 | 2 | 67 | 27 |
| 3 | 3 | 594 | 46 | 7.93 | 2 | 61 | 19 |

*only blocks of 4 SNPs or more are considered

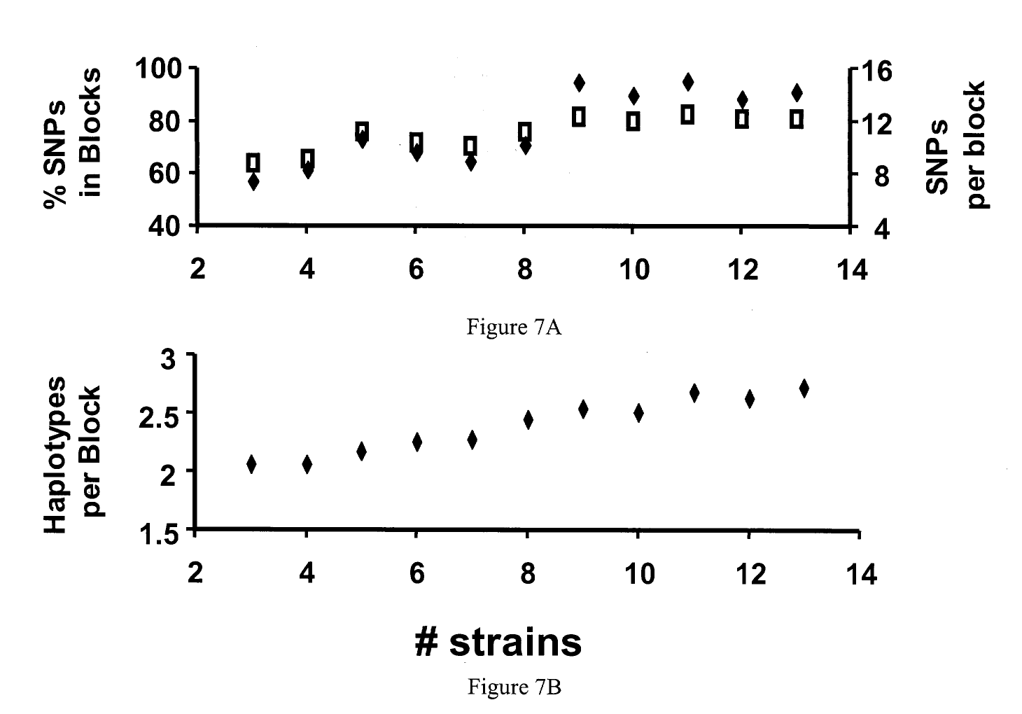

As seen in Table 3, the number of new haplotypes in each block increases only slightly as additional new strains were included in the analysis. There was an increase of 0.05 new haplotypes per strain added (FIG. 7), indicating that each additional strain usually had a pattern of polymorphism that fit within an existing haplotype within each block. The number of haplotypes within a block appeared to plateau after about 8 strains were analyzed. Across the mouse genome, over 80% of the SNPs fell into blocks containing 4 SNPs or more, and on average each block contained 14.6 SNPs and 2.7 haplotypes.

Randomization tests indicated that the haplotype block structure produced using the method comprising steps 202 through 214 of FIG. 2 resulted from a very high level of linkage disequilibrium among SNPs within haplotype blocks. For randomization, 1,270 SNPs on chromosome 1 were arranged in random order and haplotype block structures were generated using the randomly ordered SNPs. A random order for the 1,270 SNPs was generated by randomly drawing integers from the set (1,2, . . . ,1270) one at a time, until all numbers were drawn. The structure of the randomized blocks was generated by rearranging SNP allele information according to the random order, while retaining the original chromosome location. Neighboring NSPs in a block were within 1 megabase apart. This randomization process was repeated 10 times. The properties of the resulting blocks were evaluated after each iteration. When the SNP order was randomized, the percent of SNPs in blocks with at least 4 SNPs (23%±3%), and the average number of SNPs per block (5.7±0.4) was markedly decreased; and the average number of haplotypes per block (3.82±0.18) was significantly increased relative to the properly ordered SNPs. The strong contrast between the sequential and randomly ordered SNPs shows the extent of the linkage disequilibrium of murine SNPs within the same linkage group. This high level of linkage disequilibrium is a result of relatively simple genealogy of the commonly used laboratory mouse strains.

Exemplary genotypic database 52 contained 27,112 unique SNPs; and a total of 255,547 alleles generated from analysis of 15 inbred mouse strains. There were 15 different strains in exemplary genotypic database 52, and polymorphisms unique to the M. Castenius and M. Spretus strains were excluded to avoid skewing the haplotype block structures. Out of the 10,766 SNPs that were polymorphic among the 13 strains evaluated, 115 SNPs were removed because they were not biallelic, and 3,559 other SNPs were removed because there were alleles for less than 7 strains. The remaining 7,092 SNPs form 1,709 blocks; and 443 had 4 or more SNPs (containing 81% of all SNPs on chromosome 1). Haplotype blocks with at least 4 SNPs had 11.3 SNPs per block and 2.4 haplotypes per block on average, and covered 28.6 Mb of the mouse genome.

### 5.10.2 Example 2

In U.S. patent application Ser. No. 09/737,918 entitled “System and Method for Predicting Chromosomal Regions That Control Phenotypic Traits”, filed Dec. 15, 2000, and U.S. patent application Ser. No. 10/015,167 entitled “System and Method for Predicting Chromosomal Regions That Control Phenotypic Traits”, filed Dec. 11, 2001, chromosomal regions regulating complex traits could be computationally predicted by correlative analysis of phenotypic data obtained from inbred mouse strains and the extent of allele sharing within genomic regions. A determination was made as to whether the comparison of complex phenotypes to a haplotype map of the mouse genome is a better way to computationally analyzing complex traits in mice then the methods disclosed in U.S. patent application Ser. No. 09/737,918 and U.S. patent application Ser. No. 10/015,167. The correlation was determined by calculating the negative log of the ratio of the average phenotypic difference within haplotype groups relative to the phenotypic difference between haplotype groups (Equation 1) for each haplotype block in a haplotype map. The score computed using Equation 1 for each haplotype block was then adjusted based on the size and structure of the haplotype block. This process is repeated for all haplotype blocks in the haplotype map and the best matching blocks are reported.

5.10.2.1 Use Case 1 (MHC)

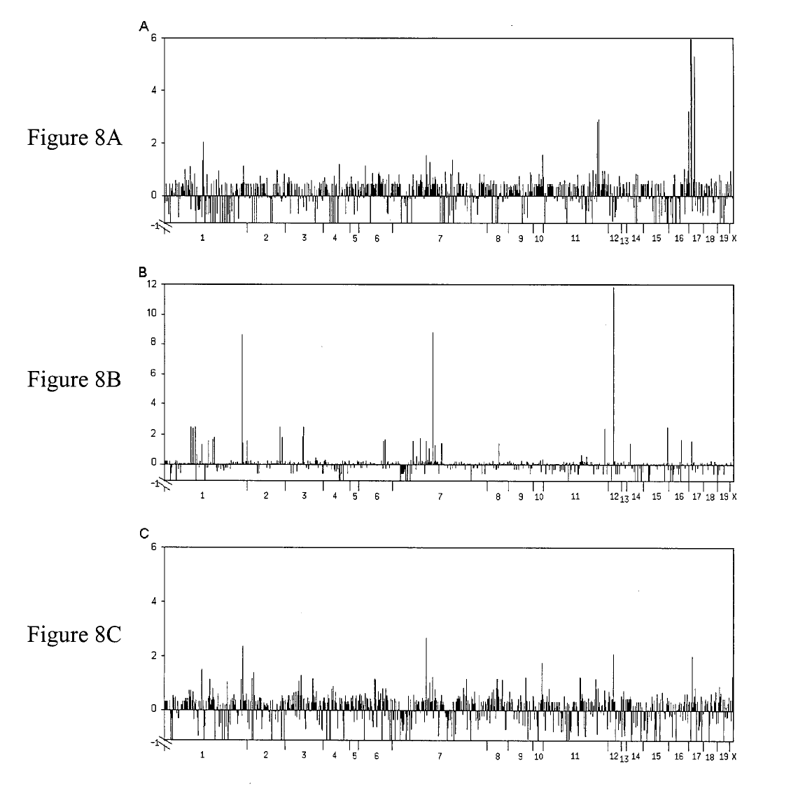

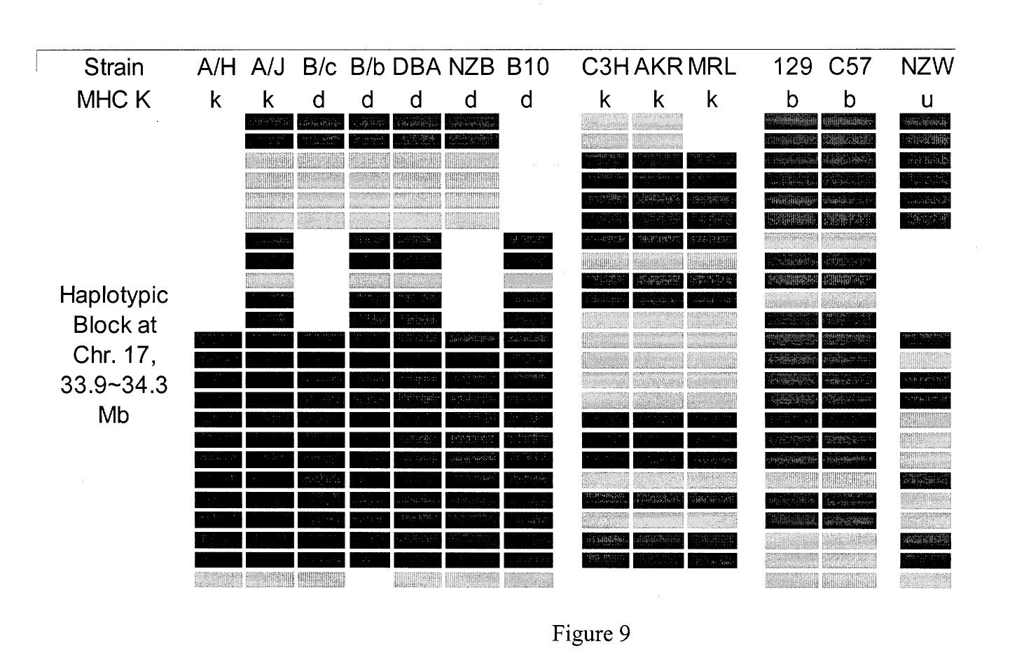

In the first use case, the haplotype-based empirical mapping method of the present invention was used to predict the chromosomal location of the K locus of the Major Histocompatibility Complex (MHC), located on murine chromosome 17 (˜33 Mb). The known H2 haplotype for the MHC K locus for 13 inbred strains was used as input phenotypic data for this analysis. The H2 haplotype of each of the 13 strains was converted to a number. Strains with the same H2 haplotype were assigned the same number. This phenotypic data was then empirically analyzed for correlation with the haplotype blocks by phenotype/haplotype processing module 44 (FIG. 1) using Equation 1 as the scoring function. As illustrated in FIG. 8A, two haplotype blocks showed a very strong correlation with the phenotypic data. In FIG. 8A, the vertical axis is standard deviation and the horizontal axis is mouse chromosome number and position. The calculated correlation was over five standard deviations above the average for all haplotype blocks analyzed. This indicated that the predicted haplotype blocks matched the phenotypic data very well (FIG. 9); and no other peaks in the mouse genome exhibited a comparable correlation with this phenotype. Both of the predicted haplotype blocks were on chromosome 17 (33.7-33.9 Mb and 33.9-34.3 Mb), and were directly adjacent to the known position of the MHC K locus. FIG. 9 illustrates the correlation between MHC K haplotype (k, d, b, u, ?) and the structure of one predicted haplotype block on chromosome 17, (33.9-34.3 megabases). Major and minor alleles are respectively indicated by dark shading and light shading whereas missing data is not shaded.

5.10.2.2 Use Case 2 (Ahr)

In the second use case, the haplotype-based empirical mapping method of the present invention was used to identify genetic loci regulating the AH phenotype (i.e., the level of induction of aromatic hydrocarbon hydroxylase activity in murine liver microsomes among inbred mouse strains). The aromatic hydrocarbon receptor (Ahr) is the ligand binding component of an intracellular protein complex that regulates the metabolism of important environmental agents, including polycyclic aromatic hydrocarbons (found in cigarette smoke and smog) and 2,3,7,8-tetrachlorodibenzo-p-dioxin (TCDD). The level of induction of aromatic hydrocarbon hydroxylase activity in murine liver microsomes (AH phenotype) varies by over 50-fold among inbred mouse strains (see Nebert et al., 1982, Genetics 100, 79-97) and this variation is thought to be due to differences in Ahr ligand binding affinity (see Chang et al., 1993, Pharmacogenetics 3, 312-321). The AH phenotype of over 40 inbred mouse strains was previously characterized (see Nebert et al., 1982, Genetics 100, 79-97); and 7 strains were in the mouse SNP database described in Example 1. The AKR/J and DBA/2J strains were AH non-responsive, while the A/J, A/HeJ, C57BL/6J, BALB/cJ and C3H/HeJ strains were AH responsive. The phenotypic response of these seven strains was evaluated with phenotype/haplotype processing module 44 (FIG. 1) using Equation 1 as the scoring function. The haplotype block containing the Ahr locus on chromosome 12 (29.6 Mb) was computationally predicted by module 44 to be the most likely region to regulate AH responsiveness (FIG. 8B), its correlation with the phenotypic data was over 10 standard deviations above the average for all haplotype blocks analyzed in this second use case. In FIG. 8B, the vertical axis is standard deviation and the horizontal axis is mouse chromosome number and position.

5.10.2.3 Use Case 3 (Cyp1a1)

Gene expression profiles across inbred mouse strains provide a useful intermediate phenotype that can be analyzed to understand how complex traits are genetically regulated. In other words, gene expression profiles can serve as phenotypic data structure 60 (FIG. 1). In the same manner as phenotypic trait information, strain-specific gene expression data can be empirically mapped onto haplotype blocks to identify genetic loci that potentially regulate differential gene expression. As one example, a cytochrome P-450 (Cyp1a1) that is required for pulmonary metabolism of xenobiotics including smoke and dioxin (see Nebert and Negishi, 1982, Biochemical Pharmacology 31, 2311-2317; Tukey et al. 1982, Cell 31, 275-284) is differentially expressed in lungs obtained from inbred mouse strains (FIG. 10A). In particular, FIG. 10A illustrates the level of pulmonary Cyp1a1 gene expression for each inbred mouse strain studied.

The data in FIG. 10A was determined as follows. Total RNA was isolated from whole mouse lung tissue. Purification of mRNA (PolyA+), synthesis of cDNA, generation of labeled cRNA and hybridization to U74v2 GeneChip© sets were performed as described in the Affymetrix Expression Analysis Technical Manual. Experiments were performed on three individual mice for each strain. Image files were generated from microarrays using four scans (HP Gene array scanner) and analyzed using MAS 5.0 software from Affymetrix, Santa Clara, Calif. To eliminate the possibility that the large number of different cytochrome genes may produce inaccuracies in the microarray data, pulmonary Cyp1a1 expression was also measured using by RT-PCR analysis, performed according to known methods. The level of expression of Cyp1a1 measured by RT-PCR analysis was completely consistent with the microarray results (data not shown).

Only 7 SNPs were identified within the entire 8-kB Cyp1a1 gene among the Mus Musculus strains analyzed. None of these SNPs were located within an exon; and the pattern of polymorphism across the strains did not correlate with the level of pulmonary Cyp1a1 expression. Therefore, the quantitatively distinct level of pulmonary Cyp1a1 expression among Mus Musculus strains was likely to be due to polymorphisms in other genes, which regulate Cyp1a1 expression in trans. For these reasons, the pulmonary Cyp1a1 gene expression data set was evaluated with phenotype/haplotype processing module 44 (FIG. 1) using Equation 1 as the scoring function. Five haplotype blocks had a significant correlation with Cyp1a1 gene expression. The haplotype block on chromo some 12 with the third highest level of correlation was the Ahr locus (FIG. 8C). In FIG. 8C, the vertical axis is standard deviation and the horizontal axis is mouse chromosome number and position. This is consistent with the known role of murine Aromatic hydrocarbon gene system in regulating the induction of numerous drug-metabolizing enzymes, including Cyp1a1 (See Nebert et al., 1982, Genetics 100, 79-87).

Polymorphisms within the Ahr locus could cause the strain-specific differential expression of Cyp1a1. The 79 SNPs identified within the Ahr locus divided the inbred mouse strains into three haplotype groups. Haplotypic group I contains the B10.D2-H2/oSnJ and C57BL/6J strains; group II contains the A/J, BALB/cJ and C3H/HeJ strains; and group III contains the 129/SvJ, AKR/J, DBA/2J and MRL/MpJ strains (FIG. 10B). A significant number of these SNPs were located in exons; producing significant changes in the amino acid sequence of the encoded protein (FIG. 1C). Four amino acid changes differentiated the group I strains from the other inbred mouse strains. One polymorphism converted a stop codon found in the group I strains (B10.D2-H2/oSnJ and C57BL/6J) to an Arg in all other strains; resulting in additional carboxyl-terminal sequence in the encoded protein. Three amino acid changes differentiated strains of group II from those of group III. One polymorphism converted a stop codon found in the group I strains (B10 and C57BL/6) to an Arg in all other strains; resulting in additional carboxyl-terminal sequence in the encoded protein. Three amino acid changes differentiated strains of group II from those of group III. One polymorphism converted an Arg in the group II strains to a Val in the group III strains. This SNP was located within a (PAC) motif that contributes to the folding of an important (PAS) domain within this protein (See Ponting and Aravind, 1997, Current Biology 7, R674-R677). The PAS domain has sites for agonist binding, as well as forming a surface for dimerization with of PAS domain containing proteins (See Burbach et al., 1992, Proceedings of the National Academy of Sciences of the United States of America 89, 8185-8189). This pattern of polymorphism and the resulting amino acid changes are consistent with the Ahr locus genetically regulating strain-specific Cyp1a1 pulmonary expression. This use case demonstrates that strain-specific gene expression data can be computationally analyzed using the systems and methods of the present invention The computational identification of a genetic locus regulating pulmonary Cyp1a1 expression, provides a first example of how gene expression data itself can be directly used for genetic analysis. Cyp1a1 is the major xenobiotic metabolizing enzyme expressed in murine (Hagg et al., 2002, Archives of Toxicology 76, 621-627) and human (Hukkanen et al., 2002, Critical Reviews in Toxicology 32, 291-411) lungs. Cyp1a1 mRNA and protein expression in murine lung was shown to increase after experimental exposure to a major environmental carcinogen (Hagg et al., 2002, Archives of Toxicology 76, 621-627). This enzyme is directly involved in the conversion of aromatic hydrocarbons, present in environmental pollutants and cigarette smoke, to active genotoxic metabolites. Therefore, it is thought to play an important role in the pathogenesis of lung cancer (Nebert, et al., 1993, Annals of the New York Academy of Sciences 685, 624-640; and Hukkanen et al., 2002, Critical Reviews in Toxicology 32, 291-411); and with cigarette smoking-associated lung diseases, such as emphysema. The computational genetic analysis in this example indicates that genetic variation within the Ahr locus regulates the basal level of Cyp1a1 expression in mouse lung.

Taken together, the three use cases in Example 2 demonstrate that the genetically regulated complex biologic processes in mice can be computationally analyzed using the haplotype map. While the techniques disclosed in U.S. patent application Ser. Nos. 09/737,918 and 10/015,167 correlated phenotypic data to chromosomal regions that were greater than twenty megabases in size, the methods of the present invention were able to predict individual genetic locus responsible for such traits, as illustrated in Example 2.

### 5.10.3 Example 3

Gene expression is normally regulated by the activity of proteins in one or more pathway(s), and multiple genes are often involved. Therefore, genetic regulation of the level of expression of a gene often results from the combined effects of polymorphisms in multiple upstream genes. Analysis of the genetic factors regulating Cyp1a1 pulmonary expression done in Example 2 illustrates how gene expression data can be used in conjunction with mapping methods of the present invention to identify genetic factors regulating a complex pathway. The computational analysis in Example 2 predicted that Ahr haplotypes regulate Cyp1a1 expression in the lung, but there may be additional levels of genetic regulation. 129/SvJ mice had a higher level of pulmonary Cyp1a1 expression than did other strains with the same Ahr haplotype (FIG. 10B; group III). This suggests that polymorphisms in another gene(s) may regulate Cyp1a1 gene expression among mice with the same Ahr haplotype. A subset of the gene expression data, constructed using only the expression data from Ahr haplotype group III strains (129/SvJ, AKR/J, DBA/2J and MRL/MpJ) (FIG. 11; step 1106) was analyzed using the methods of the present invention (FIG. 11; step 1110; see also Section 5.9). A haplotypic block containing the Arnt locus on chromosome 3 was among the top five predictions, over four standard deviations above the average (data not shown) (FIG. 11; step 1110-Yes). At the Arnt locus, 129/SvJ mice have a haplotype that clearly differentiates it from the other Ahr haplotype III strains. Arnt is known to bind Ahr and form a heterodimeric complex that regulates pulmonary Cyp1a1 transcription (Hogenesch et al., 1997, Journal of Biological Chemistry 272, 8581-8593; Reyes et al., 1992, Science 256, 1193-1195; Hoffman et al., 1991, Science 252, 954-958). This analysis suggests that the Arnt haplotype may modify the effect of Ahr haplotype in 129/SvJ mice. In the case of 129/SvJ mice, a relatively low level of pulmonary Cyp1a1 expression is expected based upon to its haplotype at the Ahr locus. However, the observed higher level of Cyp1a1 pulmonary expression in 129/SvJ mice may be due to ‘rescue’ by a high expression haplotype at the Arnt locus (FIG. 11, step 1114; Section 5.9). Although the predictions made in this example need to be independently verified, the Example indicates how the methods of the present invention using mouse haplotypes can be used to identify genetic factors regulating complex pathways.

### 5.10.4 Example 4

The present invention may be used to correlate phenotypes of a plurality of organisms of a single species with specific positions in the genome of the single species before and after the species has been exposed to a perturbation. In one implementation of this approach, two sets of experiments are performed. In the first set, the methods of the present invention are used to correlate a haplotype map to differences in a phenotype before the organisms of the single species are exposed to a perturbation. In the second set of experiments, the organisms of the single species are each exposed to a perturbation and the methods of the present invention are used to correlate a haplotype map for the species to variations in a phenotype exhibited by the organisms after they have been exposed to a perturbation. Then, the best matching haplotype blocks in the first set of experiments are compared to the best matching haplotype blocks from the second set of experiments using the methods described herein. By comparing differences or similarities between these two sets of best matching haplotype blocks, it is possible to identify regions of the genome of the single species that are highly responsive to the perturbation.

The term “perturbation” in the present invention is broad. A perturbation can be the exposure of an organism to a chemical compound such as a pharmacological or carcinogenic agent, the addition of an exogenous gene into the genome of the organism, the removal of an exogenous gene from the organism, or the alteration of the activity of a gene or protein in the organism. Thus, for example, the antibody serum level in mice representing a plurality of different mice species can be measured before and after exposing each strain of mice to an antigen. Then, the genotypic differences in the plurality of different mouse strains is correlated with observed phenotypes before and after exposure of the mice to a perturbation. By comparing the haplotype blocks that match variations in a phenotype of the mice before and after exposure to the perturbation, it is possible to localize regions of the mouse genome that are most affected by the perturbation. In some embodiments, a perturbation is a pharmacological agent. In some embodiments, a perturbation is a chemical compound having a molecular weight of less than 1000 Daltons.

Once the regions of the genome that are highly responsive to the perturbation have been identified, gene chip expression libraries that include the identified portion of the genome may be examined. Of particular interest is the identification of differential expression of genes in (i) a gene chip library made from a strain of the species before insult with a perturbation and (ii) a gene chip library made from the strain of the species after insult with a perturbation. As is well known in the art, the gene chip library may be a collection of mRNA expression levels or some other metric, such as protein expression levels of individual genes within the organism. Comparison of the differential expression level of genes in the two gene chip libraries leads to the identification of individual genes that exhibit a high degree of differential expression before and after exposure of the biological sample to a perturbation. Correlation of the positions of these individual genes with the regions of the genome identified using the correlation metrics disclosed above provides a method of identifying specific genes that are highly responsive to a perturbation.

Exemplary gene chip expression libraries have been used in studies such as those disclosed in Karp et al. “Identification of complement factor 5 as a susceptibility locus for experimental allergic asthma,” Nature Immunology 1(3), 221-226 (2000) and Rozzo et al. “Evidence for an Interferon-inducible Gene, Ifi202, in the Susceptibility of Systemic Lupus,” Immunity 15, 435-443 (2001). Furthermore, methods for making several different types of gene chip libraries are provided by vendors such as Hyseq (Sunnyvale Calif.) and Affymax (Palo Alto, Calif.).

In another approach designed to see which chromosomal regions in a genome are affected by a perturbation, phenotype data structure 60 comprises a phenotypic array for each organism in the plurality of organisms 56 in genotypic database 52 (FIG. 2) and each of these phenotypic arrays comprises a differential expression value for each cellular constituent in a plurality of cellular constituents in the organism 56 represented by the phenotypic array. In one embodiment, each differential expression value represents a difference between:

(i) a native expression value of a cellular constituent in an organism 56 in the plurality of organisms; and

(ii) an expression value of the cellular constituent in the organism 56 after the organism 56 has been exposed to a perturbation. As used herein the term “cellular constituent” includes individual genes, proteins, mRNA expressing a gene, and/or any other cellular component that is typically measured in a biological response experiment by those skilled in the art.

In some embodiments, the perturbation is a pathway perturbation. Methods for targeted perturbation of biological pathways at various levels of a cell (pathway perturbation) are known and applied in the art. Any such method that is capable of specifically targeting and controllably modifying (e.g., either by a graded increase or activation or by a graded decrease or inhibition) specific cellular constituents (e.g., gene expression, RNA concentrations, protein abundances, protein activities, or so forth) can be employed in performing pathway perturbations. Controllable modifications of cellular constituents consequentially controllably perturb pathways originating at the modified cellular constituents. Such pathways originating at specific cellular constituents are preferably employed to represent drug action in this invention. Preferable modification methods are capable of individually targeting each of a plurality of cellular constituents and most preferably a substantial fraction of such cellular constituents. See, for example, the methods described in U.S. Pat. No. 6,453,241 to Bassett, Jr., et al.

### 5.11 References Cited

All references cited herein are incorporated herein by reference in their entirety and for all purposes to the same extent as if each individual publication or patent or patent application was specifically and individually indicated to be incorporated by reference in its entirety for all purposes.

### 5.12 Alternative Embodiments

The present invention can be implemented as a computer program product that comprises a computer program mechanism embedded in a computer readable storage medium. For instance, the computer program product could contain the program modules shown in FIG. 1. These program modules may be stored on a CD-ROM, magnetic disk storage product, or any other computer readable data or program storage product. The software modules in the computer program product may also be distributed electronically, via the Internet or otherwise, by transmission of a computer data signal (in which the software modules are embedded) on a carrier wave.

Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

We claim:

1. A method of associating a phenotype exhibited by a plurality of different organisms of a single species with one or more specific genetic loci in a genome of said single species, said method comprising:
scoring a haplotype block in a haplotype map, said scoring representing a correspondence between variations in a phenotypic data structure and variations in said haplotype block, wherein
said phenotypic data structure represents a difference in said phenotype exhibited by said plurality of different organisms; and
said haplotype map includes a plurality of haplotype blocks and each haplotype block in said haplotype map represents a different portion of said genome; and
repeating said scoring for each haplotype block in said plurality of haplotype blocks in said haplotype map, thereby identifying one or more haplotype blocks in said plurality of haplotype blocks having a better score than all other haplotype blocks in said plurality of haplotype blocks; wherein said one or more specific genetic loci is each said different portion of said genome that is represented by said identified one or more haplotype blocks.

2. The method of claim 1 wherein a haplotype block in said plurality of haplotype blocks comprises a plurality of consecutive single nucleotide polymorphisms.

3. The method of claim 2 wherein each single nucleotide polymorphism in said haplotype block is within a threshold distance of another single nucleotide polymorphism in said haplotype block.

4. The method of claim 3 wherein said threshold distance is less than ten megabases.

5. The method of claim 3 wherein said threshold distance is less than one megabase.

6. The method of claim 1 wherein a haplotype block in said plurality of haplotype blocks represents a plurality of haplotypes and less than a cutoff percentage of the haplotypes represented by the haplotype block appear only once in said haplotype block.

7. The method of claim 6 wherein said cutoff percentage is in a range between five percent and thirty percent.

8. The method of claim 6 wherein said cutoff percentage is in a range between fifteen percent and twenty-five percent.

9. The method of claim 1 wherein said method further comprises the step of generating said haplotype map prior to said scoring.

10. The method of claim 9 wherein said generating comprises:
(i) identifying a candidate haplotype block having a plurality of consecutive single nucleotide polymorphisms, wherein each single nucleotide polymorphism in said candidate haplotype block is within a threshold distance of another single nucleotide polymorphism in said candidate haplotype block;
(ii) assigning a score to said candidate haplotype block;
(iii) repeating said identifying step (i) and said assigning step (ii) until all possible candidate haplotype blocks have been identified, thereby creating a set of candidate haplotype blocks;
(iv) selecting for the haplotype map a candidate haplotype block having the highest score in the set of candidate haplotype blocks;
(v) removing from said set of candidate blocks said selected candidate haplotype block and each candidate haplotype block that overlays all or a portion of said selected candidate haplotype block; and
(vi) repeating said selecting step (iv) and said removing step (v) until no candidate haplotype blocks remain in said set of candidate haplotype blocks;
wherein said haplotype map comprises each candidate haplotype block selected in an iteration of step (iv).

11. The method of claim 10 wherein said score is a number of single nucleotide polymorphisms in said candidate haplotype block divided by a square of the number of haplotypes represented by the block.

12. The method of claim 10 wherein said score is a number of single nucleotide polymorphisms in said candidate haplotype block divided by a number of haplotypes represented by the block.

13. The method of claim 1 wherein scoring said haplotype block comprises assigning a score S to said haplotype block wherein
$$S = - log ( \frac{\sum {D}_{intra}}{\sum \vert {D}_{inter} \vert} )$$
and wherein
ΣDᵢₙₜᵣₐ is a summation of the differences in phenotypic values for organisms in said plurality of organism that share the same haplotype in said haplotype block; and
ΣDᵢₙₜₑᵣ is the summation of the differences in phenotypic values between organisms in said plurality of organisms that do not share the same haplotype in said haplotype block

14. The method of claim 1 wherein scoring said haplotype block comprises assigning a score S to said haplotype block wherein
$$S = ( \frac{\sum {D}_{intra}}{\sum \vert {D}_{inter} \vert} )$$
and wherein
ΣDᵢₙₜᵣₐ is a summation of the differences in phenotypic values for organisms in said plurality of organism that share the same haplotype in said haplotype block; and
ΣDᵢₙₜₑᵣ is the summation of the differences in phenotypic values between organisms in said plurality of organisms that do not share the same haplotype in said haplotype block

15. The method of claim 1 wherein scoring said haplotype block comprises assigning a score S, wherein S is the negation, inverse, negated inverse, logarithm or negated logarithm of the ratio:
$$( \frac{\sum {D}_{intra}}{\sum \vert {D}_{inter} \vert} )$$
and wherein
ΣDᵢₙₜᵣₐ is a summation of the differences in phenotypic values for organisms in said plurality of organism that share the same haplotype in said haplotype block; and
ΣDᵢₙₜₑᵣ is the summation of the differences in phenotypic values between organisms in said plurality of organisms that do not share the same haplotype in said haplotype block

16. The method of claim 15 wherein ΣDᵢₙₜᵣₐ or ΣDᵢₙₜₑᵣ is raised to a power.

17. The method of claim 16 wherein said power is ½, 2 or 10.

18. The method of claim 1 wherein scoring said haplotype block comprises assigning a score S, wherein S is the negation, inverse, negated inverse, logarithm or negated logarithm of the ratio the ratio:
$$( \frac{\sum {D}_{intra}}{\sum \vert {D}_{inter} \vert} )$$
and wherein
ΣDᵢₙₜᵣₐ is a summation of the differences in phenotypic values for organisms in said plurality of organism that share the same haplotype in said haplotype block;
ΣDᵢₙₜₑᵣ is the summation of the differences in phenotypic values between organisms in said plurality of organisms that do not share the same haplotype in said haplotype block; and
ΣDᵢₙₜᵣₐ or ΣDᵢₙₜₑᵣ is raised to a power.

19. The method of claim 18 wherein said power is ½, 2 or 10.

20. The method of claim 1 wherein a specific genetic locus in said one or more specific genetic loci has a length that is less than 0.5 of a megabase.

21. The method of claim 1 wherein a specific genetic locus in said one or more specific genetic loci has a length between 0.5 of a megabase and 2.0 megabases.

22. The method of claim 1 wherein a specific genetic locus in said one or more specific genetic loci has a length that is less than 10 megabases

23. The method of claim 1 wherein said phenotype is diabetes, cancer, asthma, schizopherenia, arthritis, multiple sclerosis, or rheumatosis.

24. The method of claim 1 wherein said phenotype is an autoimmune disorder or a genetic disorder.

25. The method of claim 1 wherein said phentotypic data structure is microarray expression data.

26. The method of claim 1 wherein said single species is an animal, a plant, Drosophila, a yeast, a virus, or C. elegans.

27. The method of claim 1 wherein said single species is mouse or human.

28. The method of claim 1 wherein said plurality of different organisms of said single species is between five and 1000 organisms.

29. The method of claim 1 wherein said plurality of different organisms of said single species is between ten and 100 organisms.

30. The method of claim 1 wherein said plurality of different organisms of said single species is between 20 and 75 organisms.

31. The method of claim 1, the method further comprising:
(i) selecting a haplotype in said one or more haplotype blocks in said plurality of haplotype blocks having a better score than all or most other haplotype blocks in said plurality of haplotype blocks;
(ii) generating a secondary haplotype map for said single species using genotypic data for the organisms in said plurality of different organisms of said single species that are represented in said haplotype;
(iii) scoring a haplotype block in said secondary haplotype map, said scoring representing a correspondence between variations in said phenotypic data structure and variations in said haplotype block;
(iv) repeating said scoring step (iii) for each haplotype block in said secondary haplotype map, thereby identifying one or more secondary haplotype blocks having a better score than all other haplotype blocks in said secondary haplotype map; and
(v) constructing a biological pathway for said species that includes (a) a locus in the haplotype block from the haplotype block from which said haplotype was selected and (b) a locus from said one or more secondary haplotype blocks identified in an instance of step (iii).

32. The method of claim 1 wherein said phenotypic data structure represents measurements of a plurality of cellular constituents in said plurality of organisms.

33. The method of claim 1 wherein said phenotype data structure comprises a phenotypic array for each organism in said plurality of organisms and each said phenotypic array comprises a differential expression value for each cellular constituent in a plurality of cellular constituents in the organism represented by said phenotypic array, and each said differential expression value represents a difference between:
(i) a native expression value of a cellular constituent in an organism in said plurality of organisms; and
(ii) an expression value of said cellular constituent in said organism after said organism has been exposed to a perturbation.

34. The method of claim 33 wherein said perturbation is a pharmacological agent.

35. The method of claim 33 wherein said perturbation is a chemical compound having a molecular weight of less than 1000 Daltons.

36. The method of claim 1 wherein an organism in said plurality of different organisms is a member of said single species, a cellular tissue derived from a member of said single species, or a cell culture derived from said member of said single species.

37. The method of claim 1 wherein a haplotype block in said plurality of haplotype blocks comprises a plurality of restriction fragment length polymorphisms, microsatellite markers, short tandem repeats, sequence length polymorphisms, or DNA methylations.

38. A computer program product for use in conjunction with a computer system, the computer program product comprising a computer readable storage medium and a computer program mechanism embedded therein, the computer program mechanism comprising:
a genotypic database for storing variations in genomic sequences of a plurality of different organisms of a single species;
a phenotypic data structure that represents a difference in a phenotype exhibited by said plurality of different organisms;
a haplotype map that comprises a plurality of haplotype blocks, each haplotype block in said haplotype map representing a different portion of the genome of said single species; and
a phenotype/haplotype processing module for associating a phenotype exhibited by said plurality of different organisms with one or more specific genetic loci in the genome of said single species, said phenotype/haplotype processing module comprising a phenotype/haplotype comparison subroutine, said phenotype/haplotype comparison subroutine comprising:
instructions for scoring a haplotype block in said haplotype map, said scoring representing a correspondence between variations in said phenotypic data structure and variations in said haplotype block;
instructions for re-executing said instructions for scoring for each haplotype block in said plurality of haplotype blocks in said haplotype map; and
instructions for identifying one or more haplotype blocks in said plurality of haplotype blocks having a better score than all other haplotype blocks in said plurality of haplotype blocks.

39. The computer program product of claim 38 wherein a haplotype block in said plurality of haplotype blocks comprises a plurality of consecutive single nucleotide polymorphisms.

40. The computer program product of claim 39 wherein each single nucleotide polymorphism in said haplotype block is within a threshold distance of another single nucleotide polymorphism in said haplotype block

41. The computer program product of claim 40 wherein said threshold distance is less than ten megabases.

42. The computer program product of claim 40 wherein said threshold distance is less than one megabase.

43. The computer program product of claim 38 wherein a haplotype block in said plurality of haplotype blocks represents a plurality of haplotypes and less than a cutoff percentage of the haplotypes represented by the haplotype block appear only once in said haplotype block.

44. The computer program product of claim 43 wherein said cutoff percentage is in a range between five percent and thirty percent.

45. The computer program product of claim 43 wherein said cutoff percentage is in a range between fifteen percent and twenty-five percent.

46. The computer program product of claim 38 wherein said phenotype/haplotype processing module further comprises a haplotype map derivation subroutine, wherein said haplotype map derivation subroutine comprises
instructions for generating said haplotype map using said genotypic database.

47. The computer program product of claim 46 wherein said instructions for generating comprise:
(i) instructions for identifying a candidate haplotype block having a plurality of consecutive single nucleotide polymorphisms, wherein each single nucleotide polymorphism in said candidate haplotype block is within a threshold distance of another single nucleotide polymorphism in said candidate haplotype block;
(ii) instructions for assigning a score to said candidate haplotype block;
(iii) instructions for re-executing said instructions for identifying and said instructions for assigning until all possible candidate haplotype blocks in said genotypic database have been identified, thereby creating a set of undiscarded candidate haplotype blocks;
(iv) instructions for selecting for the haplotype map a candidate haplotype block having the highest score in the set of candidate haplotype blocks;
(v) instructions for removing from said set of candidate blocks said selected candidate haplotype block and each candidate haplotype block that overlays all or a portion of said selected candidate haplotype block; and
(vi) instructions for re-executing said instructions for selecting and said instructions for removing step until no candidate haplotype blocks remain in said set of candidate haplotype blocks; wherein the haplotype map comprises each candidate haplotype block selected.

48. The computer program product of claim 47 wherein said score is a number of single nucleotide polymorphisms in said candidate haplotype block divided by the square of a number of haplotypes represented by the block.

49. The computer program product of claim 47 wherein said score is a number of single nucleotide polymorphisms in said candidate haplotype block divided by a number of haplotypes represented by the block.

50. The computer program product of claim 38 wherein said instructions for scoring said haplotype block comprise instructions for assigning a score S to said haplotype block wherein
$$S = - log ( \frac{\sum {D}_{intra}}{\sum \vert {D}_{inter} \vert} )$$
and wherein
ΣDᵢₙₜᵣₐ is a summation of the differences in phenotypic values for organisms in said plurality of organism that share the same haplotype in said haplotype block; and
ΣDᵢₙₜₑᵣ is the summation of the differences in phenotypic values between organisms in said plurality of organisms that do not share the same haplotype in said haplotype block

51. The computer program product of claim 38 wherein said instructions for scoring comprise instructions for assigning a score S to said haplotype block wherein
$$S = ( \frac{\sum {D}_{intra}}{\sum \vert {D}_{inter} \vert} )$$
and wherein
ΣDᵢₙₜᵣₐ is a summation of the differences in phenotypic values for organisms in said plurality of organism that share the same haplotype in said haplotype block; and
ΣDᵢₙₜₑᵣ is the summation of the differences in phenotypic values between organisms in said plurality of organisms that do not share the same haplotype in said haplotype block

52. The computer program product of claim 38 wherein said instructions for scoring comprise instructions for assigning a score S, wherein S is the negation, inverse, negated inverse, logarithm or negated logarithm of the ratio:
$$( \frac{\sum {D}_{intra}}{\sum \vert {D}_{inter} \vert} )$$
wherein
ΣDᵢₙₜᵣₐ is a summation of the differences in phenotypic values for organisms in said plurality of organism that share the same haplotype in said haplotype block; and
ΣDᵢₙₜₑᵣ is the summation of the differences in phenotypic values between organisms in said plurality of organisms that do not share the same haplotype in said haplotype block

53. The computer program product of claim 51 wherein ΣDᵢₙₜᵣₐ or ΣDᵢₙₜₑᵣ is raised to a power.

54. The computer program product of claim 53 wherein said power is ½, 2 or 10.

55. The computer program product of claim 38 wherein said instruction for scoring said haplotype block comprise instructions for assigning a score S, wherein S is the negation, inverse, negated inverse, logarithm or negated logarithm of the ratio:
$$( \frac{\sum {D}_{intra}}{\sum \vert {D}_{inter} \vert} )$$
and wherein
ΣDᵢₙₜᵣₐ is a summation of the differences in phenotypic values for organisms in said plurality of organism that share the same haplotype in said haplotype block;
ΣDᵢₙₜₑᵣ is the summation of the differences in phenotypic values between organisms in said plurality of organisms that do not share the same haplotype in said haplotype block; and
ΣDᵢₙₜᵣₐ or ΣDᵢₙₜₑᵣ is raised to a power.

56. The computer program product of claim 55 wherein said power is ½, 2 or 10.

57. The computer program product of claim 38 wherein a specific genetic locus in said one or more specific genetic loci has a length that is less than 0.5 of a megabase.

58. The computer program product of claim 38 wherein a specific genetic locus in said one or more specific genetic loci has a length between 0.5 of a megabase and 2.0 megabases.

59. The computer program product of claim 38 wherein a specific genetic locus in said one or more specific genetic loci has a length that is less than 10 megabases

60. The computer program product of claim 38 wherein said phenotype is diabetes, cancer, asthma, schizopherenia, arthritis, multiple sclerosis, or rheumatosis.

61. The computer program product of claim 38 wherein said phenotype is an autoimmune disorder or a genetic disorder.

62. The computer program product of claim 38 wherein said phentotypic data structure is microarray expression data.

63. The computer program product of claim 38 wherein said single species is an animal, a plant, Drosophila, a yeast, a virus, or C. elegans.

64. The computer program product of claim 38 wherein said single species is mouse or human.

65. The computer program product of claim 38 wherein said plurality of different organisms of said single species is between five and 1000 organisms.

66. The computer program product of claim 38 wherein said plurality of different organisms of said single species is between ten and 100 organisms.

67. The computer program product of claim 38 wherein said plurality of different organisms of said single species is between 20 and 75 organisms.

68. The computer program product of claim 38, the phenotype/haplotype processing module further comprising:
(i) instructions for selecting a haplotype in said one or more haplotype blocks in said plurality of haplotype blocks having a better score than all or most other haplotype blocks in said plurality of haplotype blocks;
(ii) instructions for generating a secondary haplotype map for said single species using genotypic data for the organisms in said plurality of different organisms of said single species that are represented in said haplotype;
(iii) instructions for scoring a haplotype block in said secondary haplotype map, said scoring representing a correspondence between variations in said phenotypic data structure and variations in said haplotype block;
(iv) instructions for re-executing said instructions for scoring (iii) for each haplotype block in said secondary haplotype map, thereby identifying one or more secondary haplotype blocks having a better score than all other haplotype blocks in said secondary haplotype map; and
(v) instructions for constructing a biological pathway for said species that includes (a) a locus in the haplotype block from the haplotype block from which said haplotype was selected and (b) a locus from said one more or more secondary haplotype blocks identified in instances of said instructions for scoring (iii).

69. The computer program product of claim 38 wherein said phenotypic data structure represents measurements of a plurality of cellular constituents in said plurality of organisms.

70. The computer program product of claim 38 wherein said phenotype data structure comprises a phenotypic array for each organism in said plurality of organisms and each said phenotypic array comprises a differential expression value for each cellular constituent in a plurality of cellular constituents in the organism represented by said phenotypic array, and each said differential expression value represents a difference between:
(i) a native expression value of a cellular constituent in an organism in said plurality of organisms; and
(ii) an expression value of said cellular constituent in said organism after said organism has been exposed to a perturbation.

71. The computer program product of claim 70 wherein said perturbation is a pharmacological agent.

72. The computer program product of claim 70 wherein said perturbation is a chemical compound having a molecular weight of less than 1000 Daltons.

73. The computer program product of claim 38 wherein an organism in said plurality of different organisms is a member of said single species, a cellular tissue derived from a member of said single species, or a cell culture derived from said member of said single species.

74. The computer program product of claim 38 wherein a haplotype block in said plurality of haplotype blocks comprises a plurality of restriction fragment length polymorphisms, microsatellite markers, short tandem repeats, sequence length polymorphisms, or DNA methylations.

75. A computer system for associating a phenotype exhibited by a plurality of different organisms with one or more specific genetic loci in the genome of a single species, the computer system comprising:
a central processing unit;
a memory, coupled to the central processing unit, the memory storing:
a genotypic database for storing variations in genomic sequences of said plurality of different organisms of said single species;
a phenotypic data structure that represents a difference in a phenotype exhibited by said plurality of different organisms;
a haplotype map that comprises a plurality of haplotype blocks, each haplotype block in said haplotype map representing a different portion of the genome of said single species; and
a phenotype/haplotype processing module, said phenotype/haplotype processing module comprising a phenotype/haplotype comparison subroutine, said phenotype/haplotype comparison subroutine comprising:
instructions for scoring a haplotype block in said haplotype map, said scoring representing a correspondence between variations in said phenotypic data structure and variations in said haplotype block; and
instructions for re-executing said instructions for scoring for each haplotype block in said plurality of haplotype blocks in said haplotype map, thereby identifying one or more haplotype blocks in said plurality of haplotype blocks having a better score than all other haplotype blocks in said plurality of haplotype blocks.
